(19) United States
(12) Patent Application Publication
(10) Pub. No.: US 2025/0087339 A1
(43) Pub. Date: Mar. 13, 2025

(54) METHODS AND SYSTEMS FOR MODELING AND ANALYSIS

(71) Applicants: John Shen-Sampas, San Francisco, CA (US); United States Government as Represented by the Department of Veterans Affairs, Washington, DC (US); The Regents of the University of California, Oakland, CA (US)

(72) Inventors: Mark B. Ratcliffe, Piedmont, CA (US); John Shen-Sampas, San Francisco, CA (US)

(21) Appl. No.: 18/885,148

(22) Filed: Sep. 13, 2024

Related U.S. Application Data
(60) Provisional application No. 63/582,326, filed on Sep. 13, 2023.
(60) Provisional application No. 63/568,887, filed on Mar. 22, 2024.

(51) Int. Cl. G16H 30/40; G06T 7/00; G06T 7/12; G06T 7/20; G06T 17/20

(52) U.S. Cl. CPC G16H 30/40; G06T 7/0012; G06T 7/12; G06T 7/20; G06T 17/20; G06T 2207/20081; G06T 2207/30048; G06T 2210/41

(57) ABSTRACT

Methods, systems, and apparatuses are described for modeling and classifying one or more model outputs as they relate to one or more candidate objects.

### CROSS-REFERENCE TO RELATED PATENT APPLICATION

This application claims priority benefit to U.S. Provisional Application Nos. 63/582,326 and 63/568,887, filed Sep. 13, 2023 and Mar. 22, 2024, respectively, which are herein incorporated by reference in their entireties.

### BACKGROUND

Modeling and calibrating models of diastolic material properties incorporate cavity pressures, volume, strain, and other parameters but are not always successful because pressure, volume, and strain based objective functions are often indeterminate. Optimization of diastolic material properties is computationally intensive and typically requires multiple central processing unit cores and long run times.

### SUMMARY

Methods, systems, and apparatuses are described for modeling one or more objects and classifying one or more model outputs. Methods, systems, and apparatuses for optimizing one or more parameters of a constitutive equation describing the relationship between one or more material properties and/or one or more loading conditions of one or more objects (e.g., the human heart) are described. The present methods and systems leverage advanced computational algorithms and mathematical models to iteratively adjust the parameters of the constitutive equation, thereby accurately capturing the complex mechanical behavior of cardiac tissue under varying loading conditions and material properties. The systems and methods may include training a classifier and optimizing parameters based on classifier feedback. The parameters may describe stiffness or other material properties of one or more materials in one or more areas of an object (e.g., the left side of the human heart, the right side of the human heart).

Finite element analysis (FEA) is a method widely used to model and solve problems relating to complex systems such as three-dimensional non-linear design and analysis. FEA derives its name from the manner in which the geometry of the object under consideration is specified. FEA may be implemented as FEA software. The FEA software may be provided with a model and/or geometric description and associated material properties at one or more points (surfaces, areas, volumes) within the model. In this model, the geometry of the system under analysis may be represented by solids, shells and beams of various sizes, which are referred to as finite elements. The vertices of the finite elements may be referred to as nodes. The model may be comprised of a finite number of finite elements, which are assigned a material name and/or associated with material properties. The model thus represents the physical space occupied by the object under analysis along with its immediate surroundings. The FEA software may refer to a table in which the properties (e.g., stress-strain constitutive equation, Young's modulus, Poisson's ratio) of each material type are tabulated. Additionally, the conditions at the boundary of the object (i.e., loadings, physical constraints, etc.) may be specified. In this fashion a model of the object and its environment may be created.

Optimization using a biventricular (BiV) model is described. Although more difficult, use of a biventricular model may have added value especially for cardiac shape analysis because of ventricular interaction. Optimization of diastolic material properties may be achieved by using a support vector machine based classifier that defines solution feasibility. Feasibility for such a classifier may be based on heart pressures, biventricular shape metrics (e.g., a hybrid mesh shape), combinations thereof, and the like. Described herein is generation and use of a hybrid mesh comprised of tetrahedral elements at the heart apex and other difficult to mesh regions and hexahedral elements in the remainder of the heart.

FE (finite element) models of the heart may be calibrated such that the model's mechanical behavior mimics objective reality. One form of objective reality may include heart geometry measured using medical imaging (e.g., CT, MRI, ultrasound (echocardiography)) One common imaging derived geometry metric includes cavity volume. The change in cavity volume (e.g., stroke volume) and percent change (e.g., ejection fraction) during cardiac contraction (Systole) may be calculated. Another imaging based geometry metric may include regional strain. Another imaging based geometry metric may include wall thickening. Strain and wall thickening may be measured during systole, but measurement during other parts of the cardiac cycle are possible e.g., cardiac filling (e.g., diastole). Another method with which to obtain cardiac geometry/imaging based metrics may be by the analysis of heart shape. Images may be segmented into 2D contours or 3D surfaces of the outer (e.g., epicardial) or cavity (e.g., endocardial) boundaries for shape analysis that includes contour or surface axes and curvature. When comparing contours or surfaces, overlap metrics, such as the Dice coefficient (e.g., Sorensen-Dice index), may be performed. Another form of objective reality may include measured or estimated heart cavity pressures.

Model calibration may be performed by defining an objective reality to model a difference metric and minimizing the difference. The difference metrics may be referred to as an objective function (OF) and the minimization may be referred to as optimization. Model calibration may involve adjusting regional myocardial stiffness (e.g., material properties (MP)) that separately govern cardiac filling and contraction. Optimization of diastolic MP may be determined first and prior to determination of systolic MP. Previous attempts to calibrate diastolic MP incorporated cavity pressures, volume and strain in the objective function, however, those efforts were often unsuccessful because the pressure, volume and strain based objective function were indeterminate.

Optimization of diastolic material properties can be performed on a cardiac model that includes only the left ventricular (LV) chamber. However, optimization using a biventricular (BiV) model may be desirable. Although more difficult, use of a BiV model may expand opportunities for cardiac shape analysis. Specifically, the left ventricle and right ventricle may be mechanically connected through the inter-ventricular septum, and LV/RV interaction can be demonstrated through BiV shape metric analysis.

Not all combinations of material properties may lead to reasonable (e.g., feasible) deformation during filling of the heart. For instance, the inter-ventricular septum might bulge toward the left ventricle. In order to exclude unreasonable results and focus virtual experiments, optimization of diastolic material properties may be facilitated by using a classifier that defines solution feasibility. Feasibility criteria for such a classifier might be based on heart pressures or BiV shape metrics.

Not all areas of the heart have the same material properties. For instance, it is not uncommon that the left ventricle and right ventricle have different material properties (e.g., stiffness). Also, the area around the cardiac valves may be different, as would be areas that have been injured and have developed fibrosis or scar tissue. One approach to this may be to break the BiV model into parts where each part of the BiV model has its own material properties. Part specific material properties may then be separately included in the optimization. Separation of the heart model into parts might be along the lines of the 17 sector scheme proposed by the American Heart Association.

Optimization of diastolic material properties may be computationally intensive in that it typically requires multiple central processing unit (CPU) cores and long run times. As such, the use of model mesh types (such as those disclosed herein) that are both accurate and that solve in a timely manner may be advantageous. One such mesh type may include a hybrid mesh comprised of tetrahedral elements at the heart apex and other difficult to mesh regions and hexahedral elements in the remainder of the heart.

The use of a hybrid mesh may facilitate the meshing of geometrically challenging areas by meshing those areas with tetrahedral shapes, while preserving the use of more accurate hexahedral elements for the body of the Bi-V model.

The method may include generating one or more response surfaces and using one or more classifiers configured to identify one or more valleys in the one or more response surfaces. The one or more valleys in the one or more response surfaces may represent one or more combinations of variables and parameters which result in a high performing model. The optimization process may be configured to identify the one or more valleys.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

To easily identify the discussion of any particular element or act, the most significant digit or digits in a reference number refer to the figure number in which that element is first introduced.

FIG. 1 show an example system;

FIG. 2 shows an example system;

FIGS. 3A-3G show medical imaging data;

FIGS. 4A-4B show an example of mesh projection

FIGS. 5A-5C show example quadrilateral meshes;

FIG. 6 shows an example hybrid mesh;

FIG. 7 shows an example method;

FIG. 8 shows an example method;

FIG. 9 shows an example system and method;

FIGS. 10A-10B show example optimization and response curves;

FIG. 11 shows an example method;

FIG. 12 shows an example method;

FIG. 13 shows an example method; and

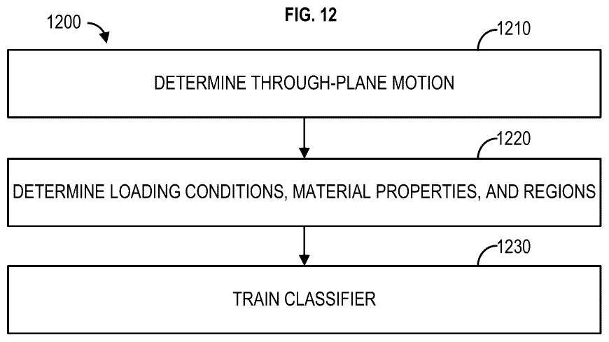

FIG. 12 shows an example system.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application, including, but not limited to, steps in disclosed methods. Thus, if there are a variety of additional steps that can be performed, it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized, including hard disks, CD-ROMs, optical storage devices, or magnetic storage devices.

Embodiments of the methods and systems are described below with reference to block diagrams and flowchart illustrations of methods, systems, apparatuses, and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The terminology used herein is for the purpose of describing particular embodiments only and is not intended to be limiting of the claims. As used in the description of the embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

As used herein, the term “user” is used interchangeably with “surgeon.” In addition, as used herein, the term “user” is also used interchangeably with “trainee,” “clinician,” “engineer,” and “scientist.”

As used herein, the term “surgical language” is used interchangeably with “medical language.” As used herein, the term “surgical language” is intended to mean the system by which physicians or surgeons or those in practice or development of medicine use to communicate or acquire thought or information. This includes letters, acronyms, words, symbols, signs, images, photographs, graphs, numbers, statistics and diagrams and other visual representations of information. It also includes sounds, tactile sensations, smell, and taste.

As used herein, the term “computational model” is intended to describe any set of mathematical equations, numerical methods, algorithms, symbolic computation, or manipulation of mathematical expressions or mathematical objects that can be used to describe or represent the physical mechanics or biology of the surgery to be studied. These models can be stochastic, deterministic, steady-state, dynamic, continuous or discrete.

As used herein, the term “medical imaging” is used interchangeably with “clinical imaging.” As used herein, “medical imaging” is intended to describe any tool and the images generated by those tools that describe or quantify anatomic features, e.g. x-rays, computed tomography (CT) scans, magnetic resonance imaging (MM), and ultrasound.

As used herein, the term “surgery” is used interchangeably with “surgical operation.”

As used herein, the term “non-surgical invasive procedure” is used interchangeably with “medical intervention.” As used herein, the term “non-surgical invasive procedure” is used interchangeably with “interventional procedures.” In addition, as used herein, the term “non-surgical invasive procedure” is used interchangeably with “minimally invasive procedure.”

As used herein, the terms “surgery” and “non-surgical invasive procedure” are intended to describe any set of actions that alters anatomy directly or indirectly. In addition, as used herein, the terms “surgery” and “non-surgical invasive procedure” include actions involved in the development of an implant, device, or product that alters anatomy directly or indirectly. An example of direct alteration of anatomy includes the use of a surgical instrument to cut or modify tissue. An example of indirect alteration of anatomy includes the use of medications that increase the strength of heart muscle contraction or increases the bone mineral density of the skeleton.

As used herein, the terms “surgery” and “non-surgical invasive procedure” are intended to describe any set of actions performed by a user that would require or be expected to require informed consent from a patient if performed or used clinically, regardless of whether or not the present use of the present disclosure is in a clinical setting.

As used herein, the distinction between a surgery and a non-surgical invasive procedure reflects the difference in visibility of the anatomy that is expected to be available to the user. In a surgery, direct visualization is used more than indirect visualization. In a non-surgical invasive procedure, indirect visualization is used more than direct visualization. In a scenario where direct visualization and indirect visualization are used equally, the user's activity is considered both a surgery and a non-surgical invasive procedure. Direct visualization reflects a direct optical pathway between the anatomy and the user and in some cases will include optics to assist with magnification or visualization. Indirect visualization reflects the use of an intermediate tool such as camera, fluoroscopy, CT, Mill or ultrasound where the user does not have a direct optical pathway to the anatomy.

As used herein, the term “solver” is used interchangeably with “solver module” and is intended to describe any set of numerical methods that are used to represent true physical phenomena such as Newton's Laws of Motion that provides sufficient accuracy to reflect clinical reality. The “finite element” approach is one such example that can be used in one embodiment of the present disclosure. The present disclosure is not restricted to the use a “finite element” based solver. In some embodiments, the solver module uses numerical methods of simulation such as finite difference, finite volume, finite element, Arbitrary Lagrangian-Eulerian, Navier-Stokes, or Conservation Element & Solution Element methodsfor fluid modeling.

As used herein, the action of “alteration of anatomy” refers to any action that can be represented as a change to a description of a geometric mesh, a material property, or any loading conditions.

As used herein, the term “geometric mesh” refers to any generated description that describes or defines the physical shape, micro- and macro-structure, or form of one or more anatomic structures.

As used herein, the term “material property” refers to any description of the physical characteristics of the anatomy described by the geometric mesh in response to physical loads. In addition, as used herein, “material property” also refers to the response to pharmacologic, electrical, magnetic, or heating or cooling interventions. In addition, as used herein, “material property” also refers to any characteristic of anatomy that can be represented as physical changes, whether directly or indirectly through biological changes.

As used herein, the term “loading condition” refers to any description of the physical loads applied to or experienced by the anatomy. In addition, as used herein, the “loading condition” includes any description of pharmacologic, electrical, magnetic, or heating or cooling interventions.

As used herein, the term “clinical information” refers to medical imaging, laboratory results such as serum potassium or calcium levels, physical examination results such as blood pressure, height, patient history such as occupation, use of tobacco products, and any additional clinical data describing a patient that can be represented as a change to the anatomy through a description of a geometric mesh, a material property, or a loading condition.

As used herein, the term “patient” refers to both an entire individual as an organism as a whole as well as any subset of the patient's anatomy such as a patient's organ system (e.g., cardiopulmonary system reflecting the heart and lungs and the associated connective tissue), an individual organ (e.g., a heart), a substructure of an organ (e.g. a heart valve), a substructure of a substructure (e.g. a leaflet of a heart valve), or a substructure of a substructure of the substructure (e.g. collagen bundle of a leaflet of a heart valve). There is no limit to the restriction to minimum size of the subset of the anatomy as the size is defined by the user's request and anatomy of interest.

Hereinafter, various embodiments of the present disclosure will be described with reference to the accompanying drawings. As used herein, the term “user” may indicate a person who uses an electronic device or a device (e.g., an artificial intelligence electronic device) that uses an electronic device.

The present disclosure provides a method used to calculate, approximate, or infer information about one or more candidate objects. The one or more candidate objects may comprise, for example, one or more organs or systems (e.g., human organs, systems, muscles, etc . . . ). This method may include appropriate adaptations of any of the varied techniques developed for functional object mapping based on medical image data, recordings, or those developed for mapping of cardiac activity based on measurements of—material properties, loading conditions, actions, and/or cardiac potentials made at the surface of the heart or the torso, or any combination of elements of these techniques.

The disclosure can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. expert inference rules generated through a neural network or production rules from statistical learning).

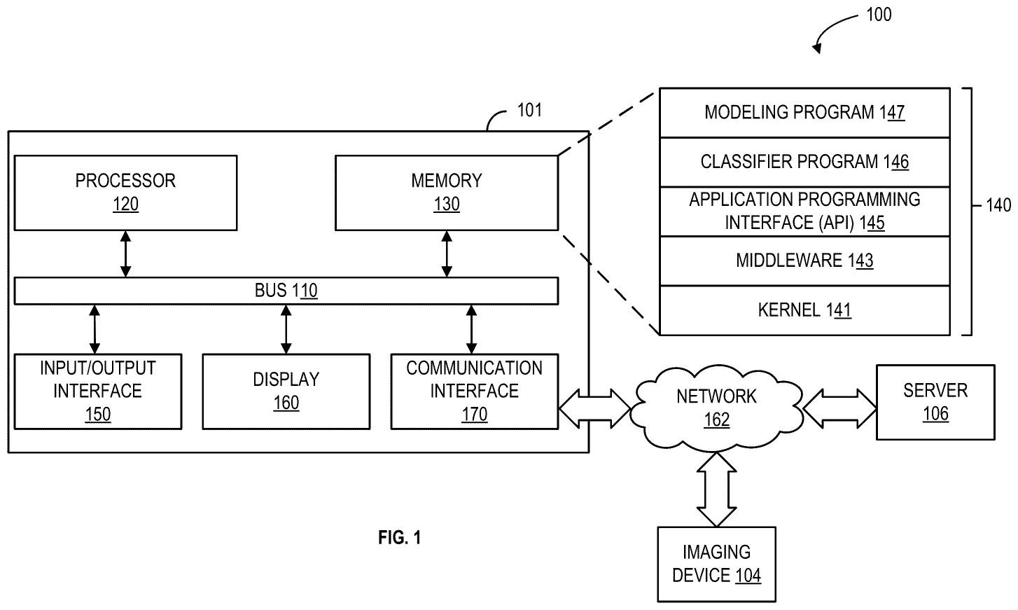

FIG. 1 illustrates a network environment including an electronic device configured for ERG signal analysis according to various embodiments. The methods and systems described herein may determine one or more parameters that describe the non-linear stiffness of the left and right sides of the heart by integrating advanced imaging techniques, finite element modeling, and optimization processes.

Referring to FIG. 1, n computing device 101 in a network environment 100 is disclosed according to various exemplary embodiments. The computing device 101 may include a bus 110, a processor 120, a memory 130, an input/output interface 150, a display 160, and a communication interface 170. In a certain exemplary embodiment, the computing device 101 may omit at least one of the aforementioned constitutional elements or may additionally include other constitutional elements. The computing device 101 may be, for example, a computer, a mobile phone, a tablet, a laptop, a desktop computer, a smartwatch, or the like. The computing device 101 may comprise or otherwise be connected to a signal processor (e.g., a signal sensing and recording device) capable of detecting and amplifying signals from an imaging device 104. The signal processor preferably is capable of processing the signals obtained from measurements in a form suitable for data analysis. The signal processor may include or can be interfaced with a data storage device (e.g., random access memory, hard drive storage, and the like) and optionally includes or can be interfaced with a display device (e.g., user interface). In some embodiments, a computer is programmed to generate a model of a candidate object.

The imaging device 104 may be configured to utilize various modalities, including but not limited to X-ray, MRI, CT, ultrasound, and PET scans. The imaging device 104 may be configured to encompass a range of imaging technologies, including X-ray, magnetic resonance imaging (MRI), computed tomography (CT), ultrasound, positron emission tomography (PET), or any combination thereof. In its embodiment, the imaging device 104 may be configured for adaptability, allowing seamless switching between different imaging modalities based on patient requirements and diagnostic objectives. For example, Cardiac MRI (CMR) captures high-resolution images of the heart, while HARP (Harmonic Phase Analysis) calculates 3D systolic strain to assess how the heart deforms during contraction. This data is supplemented by Echocardiography, including Echo-Doppler and Tissue Doppler measurements, which provide information on blood flow velocities and tissue movement, helping estimate parameters like end-diastolic pressure and volume load. Using this information, the system creates a bi-ventricular hexahedral mesh, based on the segmented left and right ventricular surfaces, and integrates rule-based fiber angles that define the muscle fiber orientation within the heart.

Furthermore, the imaging device 104 may be configured with customizable imaging protocols to cater to specific clinical scenarios and anatomical regions. These protocols encompass parameters such as scan duration, resolution, contrast settings, and anatomical coverage, all of which can be adjusted to optimize diagnostic accuracy while minimizing patient exposure to radiation or other potential risks.

In one aspect, the imaging device 104 may be configured for high-resolution imaging, utilizing state-of-the-art detectors, sensors, and signal processing algorithms to capture detailed anatomical and pathological information. This high-resolution capability enables precise visualization of structures such as bones, soft tissues, organs, and vasculature, facilitating accurate diagnosis and treatment planning.

Moreover, the imaging device 104 may be configured with advanced software functionalities, including image reconstruction algorithms, automated segmentation tools, and quantitative analysis modules. These software features enhance the diagnostic utility of the imaging device 104 by providing real-time image processing, artifact correction, and quantitative measurements, thereby improving the reliability and reproducibility of diagnostic assessments.

Additionally, the imaging device 104 may be configured with connectivity options, enabling seamless integration into existing healthcare infrastructure and electronic medical record (EMR) systems. This integration facilitates efficient data exchange, remote access, and interdisciplinary collaboration, empowering healthcare providers with timely access to imaging data and facilitating informed clinical decision-making.

The model program 147 may be configured to generate one or models of one or more candidate objects. The one or more models of the one or more candidate objects may comprise, for example, one or more mesh representations of the one or more candidate objects. The modeling program 147 may be configured as a semi-automated analysis program to perform non-subjective and repeatable feature identification (“marking”) of a candidate object.

The modeling program 147 may make use of a mesh generated based on the medical imaging data. The modeling program 147 may use the mesh in a finite element (FE) model, which simulates how the heart responds to physiological loading conditions, such as volume and pressure changes. The system applies known boundary conditions, such as end-diastolic pressure, and initially uses constitutive equations like the Ogden model to define the material behavior of the heart tissue, with parameters for stiffness and non-linear elasticity. The model then calculates the strain based on these initial parameters and compares the simulated heart deformation to the real deformation data using an inverse strain calculation.

The modeling program 147 may be configured to material parameter optimization. For example, the system may be configured to iteratively adjust one or more parameters (e.g., shape parameters, material parameters such as stiffness and non-linearity of response) for both the left and right sides of the heart. This process may continue until the simulated deformation patterns closely match the observed strain data from CMR and echocardiography. For example, the left ventricle, which may experience higher pressures, may exhibit greater stiffness and different non-linear behavior compared to the right ventricle, which has lower mechanical demands. Through this optimization process, the system may converges on the specific values for the non-linear stiffness parameters that accurately describe the tissue behavior in both ventricles. This provides a precise understanding of how stiffness varies between the left and right sides of the heart, reflecting their distinct mechanical functions.

The bus 110 may include a circuit for connecting the aforementioned constitutional elements 110 to 170 to each other and for delivering communication (e.g., a control message and/or data) between the aforementioned constitutional elements.

The processor 120 may include one or more of a Central Processing Unit (CPU), an Application Processor (AP), and a Communication Processor (CP). The processor 120 may control, for example, at least one of other constitutional elements of the Computing device 101 and/or may execute an arithmetic operation or data processing for communication. The processing (or controlling) operation of the processor 120 according to various embodiments is described in detail with reference to the following drawings.

The memory 130 may include a volatile and/or non-volatile memory. The memory 130 may store, for example, a command or data related to at least one different constitutional element of the computing device 101. According to various exemplary embodiments, the memory 130 may store a software and/or a program 140. The program 140 may include, for example, a kernel 141, a middleware 143, an Application Programming Interface (API) 145, and/or a modeling program (e.g., “application” or “mobile app”) 147, or the like. The modeling program 147 may be configured for controlling one or more functions of the computing device 101 and/or an external device (e.g., an imaging device and/or a lighting device). At least one part of the kernel 141, middleware 143, or API 145 may be referred to as an Operating System (OS). The memory 130 may include a computer-readable recording medium having a program recorded therein to perform the method according to various embodiment by the processor 120.

The classifier program 146 may be configured to classify one or more loading conditions, material properties, mechanical actions, or other properties of one or more candidate objects as biologically plausible (e.g., feasible, expected/unexpected, reasonable/unreasonable, combinations thereof, and the like) or implausible (e.g., indicative of abnormal function, infarction, etc . . . ). The classifier program 146 embodies a sophisticated computational tool tailored for image-based analysis and diagnostic decision-making in the realm of medical imaging. Specifically designed to process images of objects, such as organs or anatomical structures, the classifier program 146 utilizes advanced machine learning algorithms and predictive models to assess various input parameters and classify the results as plausible or not.

Upon receiving an image, the classifier program 146 first preprocesses and extracts relevant features from the input data, which may include anatomical landmarks, texture patterns, intensity profiles, or one or more shape descriptors. These features serve as the basis for subsequent analysis and classification tasks. For example, the one or more shape descriptors may comprise one or more mesh models. The one or more mesh models may comprise a hyrid mesh comprises of tetrahedral elements associated with one or more first regions of the candidate object (e.g., the heart apex or other difficult to mesh regions) and hexahedral elements associated with one or more second regions of the candidate object (e.g., simple regions).

The classifier program 146 may leverage a diverse set of input parameters, which may encompass quantitative measurements, imaging biomarkers, physiological parameters, or patient demographics. For instance, when analyzing an image of a heart, input parameters may include measures of muscle stiffness, myocardial perfusion, ventricular function, anatomical dimensions, combinations thereof, and the like.

Once the input parameters are collected, the classifier program 146 may employ a combination of statistical modeling, machine learning techniques, and computational simulations to generate predictive models that correlate these parameters with specific health conditions or pathological states. These models may be trained on large datasets of annotated images and clinical outcomes, enabling the classifier program 146 to learn complex patterns and relationships inherent in the data.

During the classification process, the classifier program 146 evaluates the input parameters against the trained models and computes the likelihood or probability of various diagnostic outcomes. For example, when assessing the muscle stiffness parameter, the classifier program 146 may simulate the dynamic behavior of the heart muscle using biomechanical models or computational fluid dynamics, considering factors such as cardiac contractility, myocardial compliance, and hemodynamic forces.

Based on the computed probabilities and diagnostic thresholds, the classifier program 146 may classify the results as plausible or not, providing clinicians with actionable insights and decision support for patient management. In the case of the heart image analysis, the classifier program 146 may flag abnormal muscle stiffness values indicative of myocardial infarction, cardiomyopathy, or other cardiac pathologies, prompting further investigation or intervention.

The classifier program 146 and the modeling program 147 may be in communication with (e.g., via the communication interface 170) one or more of an imaging device 104, and/or a server 106. While FIG. 1 shows one imaging device 104 and one server 106, it is to be understood that the system 100 may comprise one or more imaging devices and/or one or more servers.

The kernel 141 may control or manage, for example, one or more system resources (e.g., the bus 110, the processor 120, the memory 130, etc.) used to execute one or more operations or function implemented in other programs (e.g., the middleware 143, the API 145, or the application program 147). Further, the kernel 141 may provide an interface capable of controlling or managing the system resources by accessing individual constitutional elements of the Computing device 101 in the middleware 143, the API 145, or the application program 147.

The middleware 143 may perform, for example, a mediation role so that the API 145 or the application program 147 can communicate with the kernel 141 to exchange data.

Further, the middleware 143 may handle one or more task requests received from the modeling program 147 according to a priority. For example, the middleware 143 may assign a priority of using the system resources (e.g., the bus 110, the processor 120, or the memory 130) of the computing device 101 to at least one of the modeling programs 147. For instance, the middleware 143 may process the one or more task requests according to the priority assigned to the at least one of the application programs, and thus may perform scheduling or load balancing on the one or more task requests.

The API 145 may include at least one interface or function (e.g., instruction), for example, for file control, window control, video processing, or character control, as an interface capable of controlling a function provided by the application 147 in the kernel 141 or the middleware 143.

For example, the input/output interface 150 may play a role of an interface for delivering an instruction or data input from a user or a different external device(s) to the different constitutional elements of the Computing device 101. Further, the input/output interface 150 may output an instruction or data received from the different constitutional element(s) of the Computing device 101 to the different external device(s).

The display 160 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. The display 160 may display, for example, a variety of contents (e.g., text, image, video, icon, symbol, etc.) to the user. The display 160 may include a touch screen. For example, the display 160 may receive a touch, gesture, proximity, or hovering input by using a stylus pen or a part of a user's body.

In an embodiment, the display 160 may be configured for displaying a user interface. The user interface may be configured to receive inputs. For example, the display 160 may comprise a touchscreen. Via the user interface, a user may execute a program (e.g., a modeling or classifier program) and/or adjust loading conditions, materials properties, other parameters, combinations thereof, and the like.

The communication interface 170 may establish, for example, communication between the computing device 101 and the external device (e.g., imaging device 104, or a server 106). For example, the communication interface 170 may communicate with the external device (e.g., the imaging device 104 or the server 106) via a network 162. The network 162 may make use of both wireless and wired communication protocols.

For example, as a wireless communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), other cellular technologies, combinations thereof, and the like. Further, the wireless communication may include, for example, a near-distance communication protocol 164. The near-distance communication protocol 164 may include, for example, at least one of Wireless Fidelity (WiFi), Bluetooth, Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication may include, for example, at least one of Universal Serial Bus (USB), High Definition Multimedia Interface (HDMI), Recommended Standard-232 (RS-232), power-line communication, Plain Old Telephone Service (POTS), and the like. The network 162 may include, for example, at least one of a telecommunications network, a computer network (e.g., LAN or WAN), the internet, and a telephone network.

According to one exemplary embodiment, the server 106 may include a group of one or more servers. According to various exemplary embodiments, all or some of the operations executed by the computing device 101 may be executed in a different one or a plurality of electronic devices (e.g., the imaging device 104, or the server 106). According to one exemplary embodiment, if the computing device 101 needs to perform a certain function or service either automatically or at a request, the computing device 101 may request at least some parts of functions related thereto alternatively or additionally to a different electronic device (e.g., the imaging device 104, or the server 106) instead of executing the function or the service autonomously. The different electronic device (e.g., the imaging device 104, or the server 106) may execute the requested function or additional function and may deliver a result thereof to the computing device 101. The computing device 101 may provide the requested function or service either directly or by additionally processing the received result. For this, for example, a cloud computing, distributed computing, or client-server computing technique may be used.

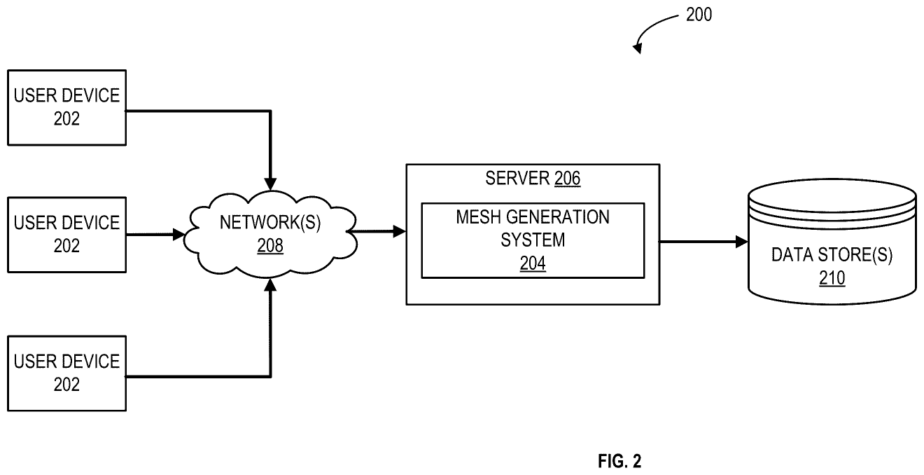

FIG. 2 depicts an example computer-implemented environment 200 wherein users can interact with a mesh-generation system 204 hosted on one or more servers 206 through a network 208. The mesh-generation system 204 can assist the users (e.g., who interact with the system via one or more user devices 202) to generate one or more meshes that include quadrilateral elements (e.g., polygons with four sides) and tetrahedral elements (e.g., polygons with three sides) to represent a physical object. For example, the generated meshes may be used for rendering physical objects, in addition to use in numerical simulations.

As shown in FIG. 2, the users can interact with the mesh-generation system 204 through a number of ways, such as over one or more networks 208. One or more servers 206 accessible through the network(s) 208 can host the mesh-generation system 204. The one or more servers 206 can also contain or have access to one or more data stores 210 for storing data for the mesh-generation system 204. The mesh-generation system 204 may configured to generate meshes through a combination of features from local quadrilateral or tetrahedral meshing and global quadrilateral or tetrahedral meshing.

According to some embodiments, the mesh-generation system 204 performs local meshing and global meshing. For example, the mesh-generation system 204 extends the local meshing approach to generate (e.g., sequentially) mesh elements using existing edges or boundaries using any advancing meshing method. In addition, the mesh-generation system 204 extends the global meshing approach to generate a directional field (e.g., a cross field) that satisfies a set of directional constraints and compute a globally smooth parameterization based on the directional field for mesh generation.

According to certain embodiments, the mesh-generation system 204 performs local meshing through a paving approach, a Q-Morph approach, or any other suitable approaches. For example, the mesh-generation system 204 adjusts certain elements to improve mesh quality and boundary smoothness. Interior angles in the paving boundary may be seamed or closed by connecting opposing elements. In addition, a next row may be adjusted to correct for elements that become too small or too large, and the paving boundary may be checked for intersections with itself or with other paving boundaries. Furthermore, the completed mesh may be adjusted (e.g., element deletion and/or addition) to improve the mesh quality.

In some embodiments, the mesh-generation system 204 extends the Q-Morph approach to utilize an existing mesh only to eliminate 3D intersection checks that result from the paving algorithm. For example, the mesh-generation system 204 may place a new point in the existing mesh and recover, through a series of splits and swaps, an edge for building the mesh. A topological clean-up and a smoothing process are performed to improve mesh element quality.

According to certain embodiments, the mesh-generation system 204 performs global meshing by constructing a directional field (e.g., a cross field) that satisfies a sparse set of directional constraints, e.g., to capture a geometric structure of a surface. For example, the directional constraints may be detected using anisotropy measures or determined by manually selection. The directional field of degree n (n is an integer) associates a collection of n evenly spaced unit tangent vectors to each point of a surface in the mesh. For instance, n=1, 2 and 4 correspond to a direction field, a line field, and a cross field, respectively. The directional field may include singularities, i.e., isolated points where the directional field does not vary smoothly. A globally smooth parametrization is computed so that iso-parameter lines follow the directional field (e.g., with n=4). Any known methods (e.g., David Bommes, Henrik Zimmer, and Leif Kobbelt, “Mixed-integer quadrangulation,” ACM Transactions on Graphics (TOG). Vol. 28. No. 3. ACM, 2009) can be implemented for performing the globally smooth parametrization. The mesh may be cut open to create a surface patch with a disk-like topology where directional field singularities lie at the boundary. Subsequently, one or more piecewise linear scalar fields are created, and the gradients of the scalar fields follow the directional field. Mesh elements (e.g., quadrilateral and/or tetrahedral) are then generated.

The mesh-generation system 204 may be configured to combine global information (e.g., a directional field and a size field) and local information (e.g., sizes of local fronts, interaction between local fronts, interactions between features, and front directions) for mesh generation. Various weights, such as a front angle weight and a length weight, may be applied for the combination of the global information and the local information. The front angle weight may correspond to an angle between the directional field (e.g., a cross field) and a front growth direction. The length weight may correspond to a ratio between the size field and a front length. For example, the mesh-generation system 204 may apply a linear combination of the front angle weight and the length weight, depending on which of the two factors (e.g., the crossfield and the size field) needs to be dominant. In another example, the mesh-generation system 204 can adopt an adaptive approach for applying the combination of the front angle weight and the length weight.

In some embodiments, for surface meshing, the mesh-generation system 204 may determine a direction most perpendicular to a current front for generating mesh elements. In certain embodiments, the mesh-generation system 204 may computer a cross field, and select one of two orthogonal directions provided by a cross field that aligns better with the front growth direction to create new fronts. For example, if the difference between the cross field and the initial front growth direction exceeds a threshold (e.g., 30°-35°, the mesh-generation system 204 may select a direction of the cross field that aligns well with one or more neighboring fronts. For example, if there is no neighboring front, the mesh-generation system 204 may determine either of the two orthogonal directions of the cross field to advance the initial front. In some embodiments, a front smoothing process may be performed (e.g., to move one or more vertices around) to improve the shapes of the generated mesh elements based on a weighted combination of a size field and a directional field (e.g., a cross field) at a front location.

In certain embodiments, the mesh-generation system 204 may generate meshes element by element. In some embodiments, the mesh-generation system 204 may generate meshes row by row. The mesh-generation system 204 may proceed from one end and mesh all the way towards the other end so as to yield well aligned meshes.

The mesh-generation system 204 can construct smooth directional fields (e.g., line fields, cross fields, etc.) that reduces a smoothness energy over configurations of singularities (e.g., number, location, and index) and aligns with a given guidance field (e.g., principal curvatures). A smoothest directional field (e.g., an optimized directional field) is may be determined using a sparse eigenvalue problem involving a matrix similar to the co tan-Laplacian matrix. With a guidance field, the optimal directional field is determined by solving a single linear system. Specifically, the mesh-generation system 204 stores n^{th} power of a complex number at each vertex of an initial mesh (e.g., a hybrid mesh, a quadrilateral mesh, a triangle mesh), together with an arbitrary (e.g., fixed) tangent basis direction. Then, the mesh-generation system 204 measures smoothness of a directional field using ground state energy of an appropriate Schrödinger operator. The mesh-generation system 204 may use a continuum of smoothness energies that provide a tradeoff between the straightness of field lines and the total number of singularities. The mesh-generation system 204 allows a tradeoff between smoothness and alignment with a guidance field (e.g., principal curvatures).

In some embodiments, the mesh-generation system 204 extends a linear system (e.g., Felix Knöppel, et al., “Globally optimal direction fields,” ACM Transactions on Graphics (TOG) 32.4 (2013): 59) with complex values for determining a globally smooth directional field (e.g., a crossfield) aligned with principal curvature directions, boundaries and/or user-specified directions. A n-vector field on a surface is represented as follows:

$$ψ = uX \qquad ( 1 )$$

where X represents one or more basis vectors and u represents a unit vector. A directional field corresponds to a unit n-vector field. A Dirichlet energy of a n-vector field is defined as follows:

$${E}_{D} ( ψ ) := \frac{1}{2} {\int }_{M} {\vert ∇ ψ \vert}^{2} dA \qquad ( 2 )$$

where ∇ represents a covariant derivative, i.e., the Levi-Civita connection on a surface M.

The covariant derivative is orthogonally split, e.g., into a sum of Cauchy-Riemann derivatives. The Dirichlet energy decomposes into a holomorphic term (E_{H}) and an anti-holomorphic term (E_{A}) as below:

$${E}_{D} ( ψ ) = {E}_{H} ( ψ ) + {E}_{λ} ( ψ ) := \frac{1}{2} {\int }_{M} {\vert \overline{\partial } ψ \vert}^{2} dA + \frac{1}{2} {\int }_{M} {\vert \partial ψ \vert}^{2} dA \qquad ( 3 )$$

A smoothness energy is defined as follows:

$${E}_{s} := ( 1 + s ) {E}_{H} + ( 1 - s ) {E}_{A} = {E}_{D} - s ( {E}_{A} - {E}_{H} ) \qquad ( 4 )$$

where s represents a parameter. The smoothness energy provides a continuum from anti-holomorphic energy (s=−1) to Dirichlet energy (s=0) to holomorphic energy (s=1). The parameter s controls the deviation of Eₛ from the standard Dirichlet energy by the difference

$${E}_{A} ( ψ ) - {E}_{H} ( ψ ) = \frac{1}{2} {\int }_{M} nK {\vert ψ \vert}^{2} dA - \frac{1}{2} {\int }_{\partial M} Im 〈 ∇ ψ , ψ 〉 \qquad ( 5 )$$

where K denotes a Gaussian curvature.

A first global minimizer of the smoothness energy Eₛ is determined by solving the problem:

$$A ψ = λψ \qquad ( 6 )$$

where λ represents a smallest eigenvalue, and A corresponds to a positive quadratic form determined based on

$${E}_{s} ( ψ ) = \frac{1}{2} 〈 〈 A ψ , ψ 〉 〉 .$$

Smoothness and alignment with a guidance field φ are to be balanced. Alignment with the field φ is accomplished via a functional

$${E}_{l} ( ψ ) = {\int }_{M} Re ( 〈 ϕ , ψ 〉 ) dA = Re ( 〈 〈 ϕ , ψ 〉 〉 ) \qquad ( 7 )$$

where φ is normalized so that ∥φ∥=1. Then,

$${E}_{s , t} := ( 1 - t ) {E}_{s} ( ψ ) - {tE}_{l} ( ψ ) \qquad ( 8 )$$

where tϵ[0,1] controls the strength of alignment.

Then, the smoothness energy E_{s,t} is minimized over all fields Ψ with ∥Ψ∥=1. The pointwise magnitude |φ| determines the local weighting between alignment and smoothness terms. A second global minimizer of the smoothness energy E_{s,t} is determined by solving the problem:

$$( A - {λ}_{t} I ) \tilde{ψ} = ϕ \qquad ( 9 )$$

where A represents a quadratic form corresponding to E_{s,t}. The result is normalized. λ₁ corresponds to the smallest eigenvalue of A, and the parameter λₜϵ[−∞, λ₁] controls the tradeoff between alignment (λₜ→−∞ for t→1) and smoothness (λₜ→λ₁ for t→0).

The continuous problem of Equation 9 is turned into a matrix problem

$$( A - {λ}_{t} M ) \tilde{u} = Mq \qquad ( 10 )$$

where q represents a coefficient vector for a piecewise linear version of the guidance field φ. The unit vector u that minimizes the discretized version of the smoothness energy Eₛ,t is given by

$$u = \frac{\tilde{u}}{‖ \tilde{u} ‖}$$

for

$$t = \frac{1}{( 1 + ‖ \tilde{u} ‖ )} .$$

For example, λₜ=0 is used as a starting value to solve the problem, based on Cholesky factorization followed by back substitution. However, the control parameter λₜ may not allow direct control on how well the resulting directional field (e.g., a crossfield) aligns with boundaries or user-specified regions.

The mesh-generation system 204 introduces constraints to the linear system related to Equation 20 and generates an overconstrained linear system. A linear least squares method is implemented to solve Au=b, with A=(A_{F} Ac) (a square matrix),

$$u = ( \begin{matrix}{u}_{F} \\ {u}_{C}\end{matrix} ) ,$$

b=Mq and

$$b = ( \begin{matrix}{b}_{F} \\ {b}_{C}\end{matrix} ) .$$

To balance global alignment (e.g., with principal curvature directions) and local alignment (e.g., with boundaries and/or user-specified directions), a parameter a is introduced into the system represented by Equation 10.

Au=αMq is to be solved with A=(A_{F} A_{C}) (a square matrix),

$$u = ( \begin{matrix}{u}_{F} \\ {u}_{C}\end{matrix} ) .$$

A_{F} represents a rectangular matrix of size (N_{F}+N_{C}, N_{F}), and A_{C} represents a rectangular matrix of size (N_{F}+N_{C}, N_{C}), where N_{C} represents a number of constraints and N_{F} represents a number of unknowns. Thus, A_{F}u_{F}=αMq−A_{C}u_{C}.

Let b=αMq−A_{C}u_{C}. Then, A_{F} ^{t}A_{F}u_{F}=A_{F} ^{t}b, where A_{F}^{t} represents a complex transpose matrix (or hermitian) of A_{F}. Then, Bu_{F}=b′ is to be solved, with B=A_{F} ^{t} A_{F} (a square matrix) and b′=A_{F} ^{t}b′.

In some embodiments, the constrained degrees of freedom take pre-specified values and the system is calculated using a least-squares approach. The computed directional field is aligned with principal curvature directions of an input geometric structure. In addition, the computed directional field is also assigned with boundaries (e.g., holes, cutouts, outer edges of a surface, etc.), sharp features (e.g., crease edges, etc.), prominent features (e.g., features with high curvatures, smooth creases, etc.), and/or user-specified directions.

The extension of the linear system, as described above, is merely an example, which should not unduly limit the scope of the invention. Any cross-field design methods (e.g., iterative and/or matrix solutions) may be implemented. In some embodiments, constraints can be added in an n-angle space. For example, directional constraints can be applied to any algorithms that calculate a directional field (e.g., a crossfield) as a system of linear equations.

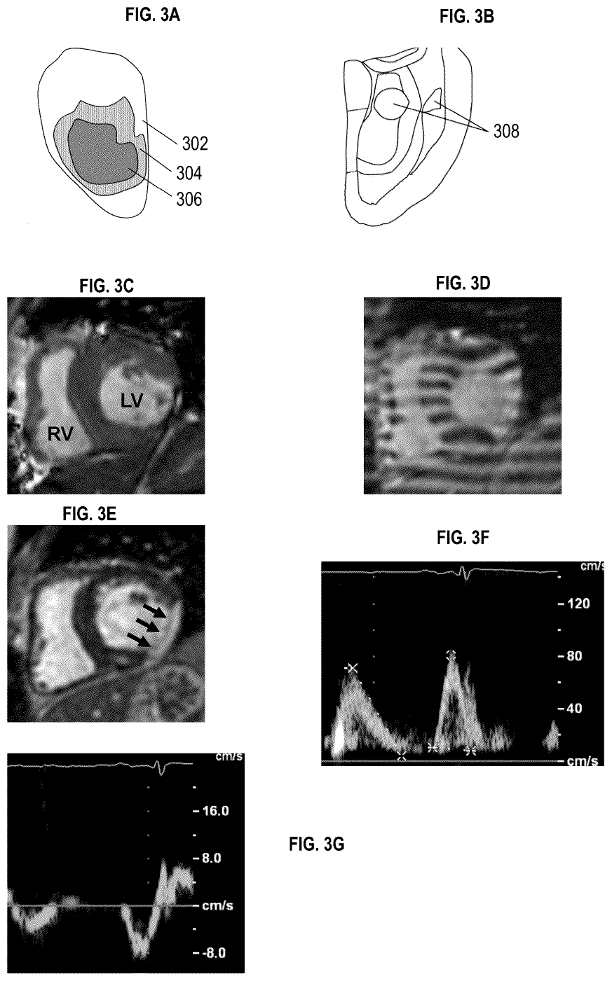

FIGS. 3A and 3B are exemplary drawings of the various aspects of a real heart, in accordance with various embodiments of the present disclosure. FIG. 3A shows the outside of the left ventricle and in this case also shows an area of heart attack (myocardial infarction (MI) where the MI (306) is surrounded by a border of poorly contractile heart muscle (304) and the normal undamaged heart muscle (302). FIG. 3B shows the inside of the left ventricle. The mitral valve is the inflow valve to the left ventricle and FIG. 3B further shows the muscular connections or papillary muscles (308) by which the valve is connected to the left ventricular wall.

FIGS. 3C-3G show examples of the kind of imaging data that might be used to inform the shape metric process. FIGS. 3C-3E are MRI images. MRI data may be used to generate a geometric mesh. For example, preprocessing steps such as noise reduction and image smoothing are applied to enhance the quality of the images, making it easier to define the boundaries of anatomical structures. Following this, segmentation is performed to isolate specific tissues or structures, like the heart's ventricles and valves. Segmentation can be done manually or with the help of semi-automatic tools available in software like Mimics, 3D Slicer, or Simpleware, which enable accurate labeling of different regions of the organ.

After segmentation, the 2D slices are combined to create a 3D volumetric model, which represents the geometry of the organ in continuous form. This 3D model, typically saved in formats like STL, defines the external surfaces of the segmented structure. Once the model is created, the next step is to generate a finite element mesh by breaking down the geometry into smaller elements, such as tetrahedrons or hexahedrons, which can be used for simulation purposes.

Once the mesh is generated, material properties reflecting the mechanical behavior of the tissues are assigned to different regions of the model. For example, the myocardium (heart muscle) and valves may have distinct properties like elasticity and stiffness. After assigning material properties, boundary conditions and loading forces, such as muscle contraction or blood pressure, are applied to the model.

FIG. 3C is conventional cine cardiac MRI. FIG. 3C shows a biventricular image of the heart with the right ventricle (RV) and left ventricle (LV) labeled. The present methods and systems may make use of a BiV model of the candidate object (e.g., the heart) to increase the accuracy of a trained classifier in determining normal or abnormal heart function. For example, using a bi-ventricular model typically includes different loading conditions for the left and right ventricles due to their different functions (the left ventricle pumps blood to the body, and the right ventricle pumps blood to the lungs). This means that the constitutive model must be able to handle the different pressures and mechanical behaviors associated with each ventricle. Thus, the present methods and systems may consider terms for anisotropic behavior, fiber orientation, and layer-specific properties associated with the RV and LV. For example, implementing the Ogden model (as described in greater detail below) in a finite element analysis (FEA) software like LS-Dyna for a bi-ventricular heart model may include defining the hybrid mesh with detailed geometry of both ventricles, and assigning region-specific material parameters to account for the differences in tissue properties between the left and right ventricles.

FIG. 3D is an example image of cardiac deformation. The alternating light and dark bands indicate myocardial strain analysis or tissue tagging to track the heart's motion during contraction and relaxation. These bands indicate specific points in the myocardium that are tracked to observe deformation over time, illustrating how the heart contracts and expands throughout the cardiac cycle. The central lighter region represent scar tissue or an area affected by infarction, which may be stiffer and does not deform in the same way as the surrounding healthy tissue, which displays the typical striation pattern indicative of normal movement. This type of imaging may be used in the present systems and methods to evaluate the heart's mechanical function, identifying abnormalities in motion, such as reduced movement in scarred regions, and assessing overall heart health. This type of image data may be used in the present systems and methods to determine how the stress-strain relationship in the heart is altered due to damaged or compromised tissue.

FIG. 3E is an example of image scar from a medical image which may be incorporated into the present systems and methods inform the material properties of one of the sectors of a candidate object. Scar tissue in an organ (e.g., the heart) may impair heart function. Scarring in the heart, such as that caused by a myocardial infarction, significantly impacts the non-linear relationship between stress and strain due to the altered mechanical properties of the tissue. Scar tissue is much stiffer and less elastic than healthy myocardial tissue, which disrupts the normal non-linear stress-strain behavior. In healthy heart tissue, the stress-strain relationship is characterized by greater flexibility at low strains, with a progressive stiffening as strain increases. However, scar tissue exhibits a more linear and rigid response, deforming less even under high stresses. This results in a steeper stress-strain curve in scarred regions, making them less compliant compared to the surrounding healthy tissue.

The presence of scarring creates heterogeneity in the stress distribution across the heart. Healthy tissue, which retains its anisotropic and elastic properties, must compensate for the reduced flexibility of scarred areas. This leads to stress concentrations at the boundaries between scarred and healthy tissue, causing the healthy regions to experience higher strains under the same loading conditions. Over time, this can accelerate damage or remodeling in these healthy regions. Additionally, the stiff scar tissue reduces the heart's overall compliance, particularly during diastole, impairing its ability to expand and fill with blood, which may contribute to diastolic dysfunction.

When scarring is present the heart's overall stress-strain relationship becomes a mix of non-linear and more linear behavior, with non-linear elasticity dominating in the healthy myocardium and linear-like stiffness characterizing the scarred areas. This flattening of the stress-strain curve reduces the heart's contractility and overall pumping efficiency, particularly if scarring affects critical regions like the left ventricle. In terms of energy distribution, the scarred tissue stores less strain energy due to its inability to stretch, further decreasing the heart's mechanical efficiency. Over time, this increased load on the healthy tissue can lead to hypertrophy, altering the global stress-strain relationship of the heart.

The present methods and systems may incorporate such variations in material properties in constitutive equations like the Ogden model which can be adjusted to represent scarring by modifying the material parameters in the scarred regions. Stiffness parameters may increase, and the non-linear response may become more linear, reflecting the reduced compliance and altered mechanical behavior of the scarred tissue. This modification allows for the simulation of the heart's response in both healthy and scarred regions, capturing the mechanical impact of scarring on heart function. For example, the strain energy function for scar tissue might be altered to reflect a nearly linear stress-strain behavior:

$${W}_{scar} = {\sum }_{p = 1}^{N} \frac{{μ}_{p , scar}}{{α}_{p , scar}} ( λ \text{?} + λ \text{?} + λ \text{?} - 3 )$$

$$\text{?} \text{indicates text missing or illegible when filed}$$

Where μ_{p,scar} is larger and α_{p,scar} is close to 1 in scar tissue regions compared to healthy myocardium.

FIG. 3F is the signal obtained by imaging blood flow across the mitral valve. The Doppler signal provides critical information about blood flow velocities in the heart, which can be used to inform both the constitutive model of heart tissue and the classifier that distinguishes between normal and abnormal heart function. The velocity peaks and patterns during different phases of the cardiac cycle, such as systole and diastole, can be incorporated into the constitutive equation to refine the boundary conditions and loadings. This helps ensure that the model accurately reflects how the heart tissue deforms in response to blood flow. Abnormal flow patterns, which may indicate conditions like heart failure or valve dysfunction, can prompt adjustments in the material properties of the model, such as increasing stiffness to represent fibrotic or compromised tissue.

The Doppler data can also be used as a feature input for the machine learning classifier. By analyzing characteristics such as peak velocities, waveform shapes, and deceleration times, the classifier can be trained to recognize patterns associated with normal and abnormal heart function. For example, specific flow abnormalities seen in cases of diastolic dysfunction or valvular disease would be recognized by the classifier as indicators of heart dysfunction. Based on these insights from the Doppler data, the constitutive equation can be further modified to account for the mechanical effects of abnormal blood flow, improving the model's accuracy in both normal and pathological conditions. In summary, Doppler signals are essential for refining both the constitutive model and the classifier, enabling a more accurate simulation of heart tissue mechanics and a more precise identification of abnormal heart function.

FIG. 3G is the signal obtained by imaging the muscle at the base of the heart. Echocardiography is a non-invasive medical imaging technique that uses ultrasound waves to create detailed, real-time images of the heart. It may be used to assess the heart's structure, size, shape, and overall function, including how well the heart is pumping blood, the condition of its valves, and the movement of blood through the heart chambers. Echocardiograph typically works by using a transducer, which emits high-frequency sound waves toward the heart. These sound waves bounce off the heart's structures and are reflected back to the transducer, which sends the data to a computer that processes the information and generates moving images of the heart in real-time.

There are several types of echocardiography, each designed for specific clinical purposes. The most common type is the transthoracic echocardiogram (TTE), in which the transducer is placed on the chest wall to capture images of the heart. Transesophageal echocardiography (TEE) involves placing the transducer in the esophagus, providing clearer images of the heart, particularly when detailed views of the heart valves or the back of the heart are needed. Stress echocardiography is performed while the heart is under stress, either through exercise or medication, to evaluate how the heart performs under physical exertion, often used to detect coronary artery disease. Doppler echocardiography measures the speed and direction of blood flow through the heart, identifying abnormal patterns such as valve regurgitation or blockages. 3D echocardiography offers detailed, three-dimensional images of the heart, useful for assessing valve structure and function, as well as for surgical planning.

The echocardiography data may be used to generate one or more geometric meshes. 3D or 4D echocardiographic data is acquired, capturing the heart's structure and function in real-time. The data then undergoes segmentation, a process where the boundaries of different heart structures, such as the ventricles, atria, and valves, are identified and isolated. This can be challenging due to the noisier and lower-resolution nature of ultrasound images, but segmentation software, such as EchoPAC or 3D Slicer, can assist in delineating these structures. After segmentation, the 2D slices of echocardiographic images are reconstructed into a 3D model of the heart. This step involves interpolating the segmented data into a continuous 3D geometry that defines the heart's surfaces. Meshing tools like Simpleware or ANSYS may be used to break down the 3D model into smaller elements, such as tetrahedral or hexahedral elements, forming a finite element mesh. Each region of the heart, such as the myocardium or valves, is then assigned material properties that reflect the mechanical behavior of the tissue.

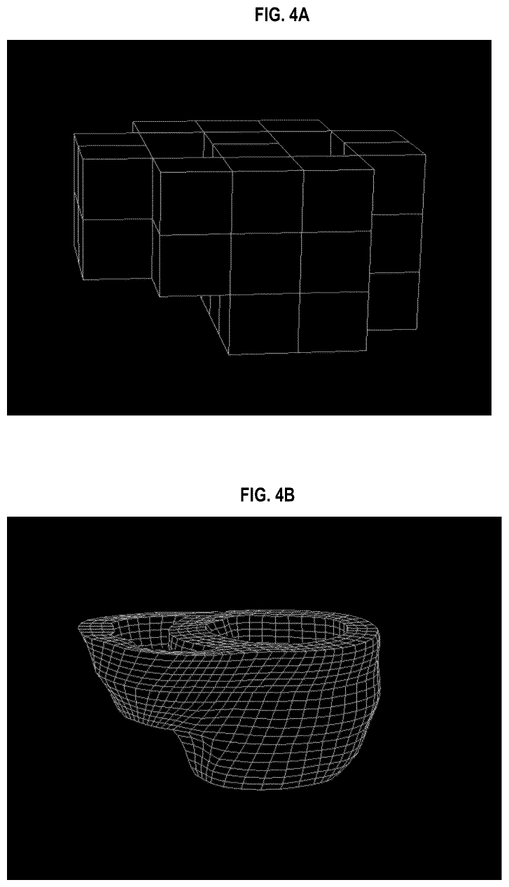

FIG. 4A shows a block mesh template. The block mesh template may comprise a regular, structured grid comprising one or more elements (e.g., cubic elements, though other elements are contemplated). The block mesh template may be generated, for example, in a Cartesian coordinate system, though other coordinate systems are contemplated. The example block mesh template may comprise one or more elements which are uniformly shaped and spaced. Block mesh templates comprising non-uniform shapes and spacing are also contemplated. The block mesh template may be configured for manipulation for further refinement and projection onto more complex geometries, such as anatomical structures like the heart. For example, the block mesh template may be configured to facilitate modification of the size (e.g., volume) of the one or more elements. The block mesh template may be configured to facilitate modification of the topology or other aspects of the block mesh template. For example, the one or more elements may be manipulated to match the curvature of a more complex structure (e.g., the structure of the candidate object).

The block mesh template (and resultant mesh of the target object after projection) may comprise one or more surfaces and/or one or more nodes. For example, a node may comprise a fundamental point in a mesh configured for use in computational models like finite element analysis (FEA). For example, the one or more nodes may comprise one or more discrete points that define the geometry and topology of the structure being modeled. The one or more nodes may serve as connection points between mesh elements (e.g., surfaces such as triangles, quadrilaterals, tetrahedrons, or hexahedrons), which together form the mesh that represents the surface or volume of the object being simulated.

The one or more nodes may specify the spatial location of the mesh in 2D or 3D space. For example, in a 3D model, each node is defined by its coordinates (e.g., x, y, z). The one or more nodes may be configured and/or arranged to form the vertices of the mesh elements (e.g., triangles, quadrilaterals, tetrahedrons). Each element of the one or more elements may be defined by (e.g., consists of) one or more nodes at its corners. For example, a quadrilateral element may four nodes, while a triangle may have three nodes.

The one or more nodes may be associated with (e.g., configured to store) information (e.g., one or more variables). For example, each nodes of the one or more nodes may be associated with stress data, strain data, displacement data, temperature data, or other properties (e.g., physical properties).

The one or more surfaces may also be associated with various information. For example, the one or more surfaces may also be associated with one or more physical properties. For example, the one or more surfaces may be associated with density information, stiffness information, deformation behavior information, conductivity, thermal capacity, temperature information, combinations thereof, and the like.

FIG. 4B shows the result of the projection method which projects the block mesh template onto the target geometry (e.g., the target object). The target geometry may be determined based on, for example, medical imaging data (e.g., MRI, CT, echocardiography methods as described herein). The target geometry may be generated by and/or imported from a CAD/CAM system. An initial crude approximation of the mesh may be generated. For example, the initial mesh may comprise only rectangular surfaces. The crude mesh may be projected onto the complex geometry and fitted appropriately. After initial projection, the resultant mesh may have imperfect geometry. For example, the mesh may include one or more gaps, one or more overlaps, combinations thereof, and the like. The method may comprise determining the gaps and/or overlaps and addressing those errors by, for example, combining one or more surfaces and/or filling in any gaps by extrapolation. The method may address imperfections like gaps and overlaps by ignoring boundaries and automatically locating surface intersections. This approach simplifies mesh creation, even for complex geometries, ensuring high-quality meshes without the need for detailed input, and can also precisely place mesh lines along critical intersections.

The projection method may comprise projecting the block mesh template onto a more complex target structures such as the candidate object (e.g., the heart or some other object). The method may comprise receiving a target object geometry. The target object geometry, such as a heart or section thereof, may be determined based on imaging data. The target geometry may be defined as a surface mesh or a 3D set of points representing, for example, an anatomical shape. The method may comprise mapping the nodes of the block mesh onto a more complex target geometry. For example, each node from the structured grid is projected onto the surface of the more intricate anatomical model by determining the closest corresponding point on the target surface. The cubic elements of the block mesh may be manipulated or changed (e.g., warped, deformed, changed in size, shape, volume, etc.) in order to align with the underlying geometry, adapting to the curved and irregular surfaces of the anatomy, such as the left and right ventricles of the heart.

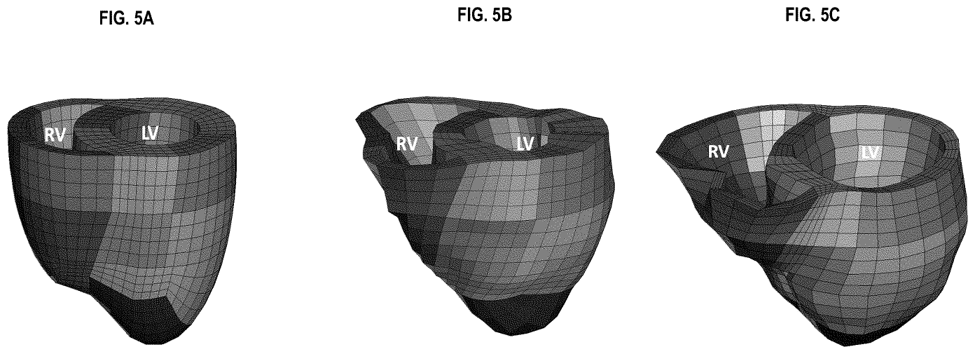

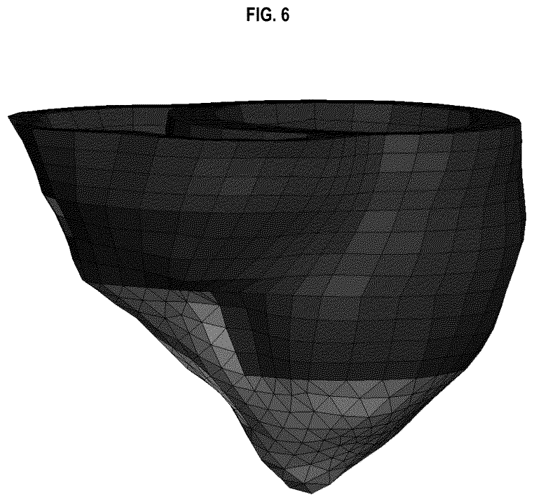

FIGS. 5A-5C show exemplary mesh representations of a heart. Separation of the heart model into parts might be along the lines of the 17 sector scheme proposed by the American Heart Association. To generate the one or more mesh representations, a user provides patient information including cardiac pressures and imaging data to the computing device. Once the data is received by the computing device, a computational model is created. In some embodiments, the computational model is a finite element model. A prototypical source model, e.g., a three-dimensional (3-D) computational model representing a physical object, such as an individual patient or a hypothetical patient made from averages. In some embodiments, the 3-D model contains one or more geometric meshes, material properties, and loading conditions. In some embodiments, parametric analysis is performed by changing the mesh geometry (altering the shape of the heart) or adding or removing the mesh geometry. In some embodiments, changing the assigned material properties (altering the stiffness/softness of the tissue) is performed. In some embodiments, changing the loading conditions (e.g. blood pressure) is performed. In some embodiments, small, directional increments in the geometric mesh, material properties, or loading conditions are performed. Parametric analysis in this way allows for understanding, in general terms, of the consequences or benefits of an action. For instance, if a heart is to be resized, the starting point would be a normal heart. Then, incrementally, the heart is adjusted to be smaller and smaller or bigger and bigger. Various surgical operations and other non-surgical invasive procedures and devices can be simulated in this way.

The geometric mesh is then assigned material properties based upon both values in the Mill and a known database of materials in clinical translation module. The age, blood pressure and current medications are used to further refine the material properties and establish the loading conditions for the computational model. This process generates a finite element model which is then shown to the surgeon via a user interface.

In some embodiments, clinical imaging data available lacks sufficient visual fidelity to identify anatomic structures of importance. In some embodiments, the information integration module will generate a geometric mesh, material property, or a loading condition representing structures that are known to exist despite the absence of the structure on imaging based upon landmarks that are visible. In some embodiments, the addition of computational models of anatomic structures not visible in the imaging data occurs without additional user input. In other embodiments, the addition of computational models of anatomic structures not visible in the imaging data occurs with user input.

In some embodiments, clinical information necessary for a computational model used by the classifier is not available. One such example would be a user that has not specified the blood pressure, or a height, or a weight as input. In some embodiments, the classifier or another component may provide the missing clinical information necessary for a computational model used by the classifier. In some embodiments, the substituted information reflects a 50th percentile value. In some embodiments, the substituted information reflects a 5th percentile value. In some embodiments, the substituted information reflects a 95th percentile value. In some embodiments, the substitution of clinical information is performed without additional user input. In other embodiments, the substitution of clinical information occurs with user input.

In some embodiments a template computational model represents an average patient. In other embodiments, the template computational model represents a patient with anatomy that does not have an average geometric mesh, material property, or loading condition. One such example would be a patient with a disease.

In some embodiments, the user will alter a material property directly to determine the effects of that action. In some embodiments, the alteration is done using surgical language. In some embodiments, the alteration is done through manipulation of a material property directly through the user interface.

In some embodiments, the user will alter a loading condition directly to determine the effects of that action. In some embodiments, the alteration is done using surgical language. In some embodiments, the alteration is done through manipulation of a loading condition directly through the user interface.

FIG. 5A shows a model which is not realistic and does not accurately represent the underlying object. FIG. 5A does show the standard 17 section model of the heart using hexahedral units (e.g., “bricks”) to model the heart. FIG. 5B shows a mesh representation of a standard heart. Again, the standard 17 sections are comprised of one or more hexahedral brick components. FIG. 5C shows a mesh representation of a heart that has suffered a heart attack.

FIG. 6 shows an example hybrid mesh representation of an object. In computational modeling of the heart, both tetrahedral and hexahedral elements may be utilized to discretize the heart's volume for simulation purposes. Tetrahedral elements, characterized by their simplicity with four vertices and four triangular faces, excel in representing irregular shapes and complex geometries. They are particularly valuable in simulations requiring accurate depiction of irregular boundaries, such as in computational fluid dynamics (CFD) studies of blood flow within the heart chambers. However, tetrahedral meshes may necessitate more elements for comparable accuracy to hexahedral meshes, potentially leading to higher computational costs. On the other hand, hexahedral elements, defined by their structured nature with eight vertices and six quadrilateral faces, offer advantages in scenarios where regularity and alignment with the underlying structure are critical, such as modeling muscle fibers within the heart. Despite potentially lower computational costs due to requiring fewer elements, hexahedral meshes might struggle to accurately represent regions with significant curvature or irregular geometries.

The present methods and systems may determine whether to use tetrahedral or quadrilateral elements in a hybrid mesh based on several factors, including the complexity of the heart's geometry, the surface curvature, and the need for accuracy in specific regions. Tetrahedral elements may be applied to areas with more complex or irregular shapes, such as regions with high curvature or intricate anatomical details like the apex of the ventricles or the boundaries between chambers. These tetrahedral components are well-suited for capturing the intricacies of 3D shapes and ensuring accuracy in regions where the heart experiences rapid changes in geometry.

In contrast, quadrilateral elements may be used for smoother or less complex areas of the heart, such as the flatter walls of the ventricles. These elements can provide greater computational efficiency in regions with more uniform geometry, where fewer shape adjustments are necessary. The system may analyze the surface curvature from the medical imaging data and assign tetrahedral elements to regions with high curvature or significant anatomical variability, while reserving quadrilateral elements for flatter, less variable surfaces.

Optimization of diastolic MP may be computationally intensive in that it typically requires multiple CPU cores and long run times. As such, the use of model mesh types that are both accurate and that solve in a timely manner may be advantageous. One such mesh type may include a hybrid mesh comprised of tetrahedral elements at the heart apex and other difficult to mesh regions and hexahedral elements in the remainder of the heart.

The use of a hybrid mesh may facilitate the meshing of geometrically challenging areas by meshing those areas with tetrahedral, while preserving the use of more accurate hexahedral elements for the body of the Bi-V model.

A mesh corresponds to a discretization of the geometrical domain and includes a collection of mesh entities. The mesh entities often have simple shapes. For example, zero-dimensional mesh entities include vertices, one-dimensional mesh entities include lines, two-dimensional mesh entities include triangles or quandrangles, and three-dimensional mesh entities include tetrahedra, hexahedra or prisms.

According to one embodiment, a method for generating one or more hybrid mesh elements (e.g., one or more tetrahedral mesh elements and/or one or more hexahedral mesh elements) to represent a physical object includes: receiving a geometric structure representing a physical object, a first data structure for the geometric structure being stored in a non-transitory computer-readable storage medium; determining a directional field using one or more data processors, data related to the directional field being stored in the non-transitory computer-readable storage medium; determining a size field using the one or more data processors, data related to the size field being stored being stored in the non-transitory computer-readable storage medium; selecting one or more locations from a region of the geometric structure, local data associated with the locations being stored in the non-transitory computer-readable storage medium; and generating one or more mesh elements based at least in part on the directional field, the size field, and the local data using the one or more data processors, a second data structure for the one or more mesh elements being stored in the non-transitory computer-readable storage medium. The second data structure is updated based at least in part on the first data structure, the data related to the directional field, the data related to the size field and the local data.

The present methods and systems use cardiac shape analysis-based metrics in the objective function of a cardiac material property optimization to produce a constrained converged solution with accurate determination of cardiac MPs. The method may use a classifier to define solution feasibility. The classifier is based in part on cardiac shape analysis-based metrics. The method may use a biventricular finite element model. The method may optionally break the model into separate parts such the left ventricle, right ventricle, interventricular septum, area around cardiac valves and areas of cardiac injury such as an myocardial infarction scar or fibrosis where each part has its own material properties. The BiV model may be meshed using both tetrahedral and hexahedral elements (Hybrid mesh). The heart moves in the chest during a heartbeat and thus moves through an imaging coordinate system. This is called “through plane motion” and finite element model may translate such through-place motion so as to mimic through plane motion as a component of the present systems and methods as it allows accurate comparison of imaging and model geometry. The method can be generalized to include multiple time points through the cardiac cycle and multiple 2D slices or 3D volumes. The inclusion of additional data may in fact be more accurate and robust.

Additionally, the methods and systems may take into account regions that are expected to experience higher stress or strain, such as areas near scar tissue or the junctions between different types of tissue. In these cases, finer tetrahedral meshing may be applied to capture localized mechanical effects with higher precision. Conversely, quadrilateral elements may be utilized in regions where stress and strain are more uniformly distributed, allowing for a coarser mesh without sacrificing the accuracy needed for the simulation.

To ensure a seamless transition between tetrahedral and quadrilateral elements, the present methods and systems may define transition zones where the two mesh types blend smoothly, maintaining the accuracy required for complex areas while optimizing the mesh in simpler regions. Mesh density could be adjusted automatically based on the complexity of the surface and the mechanical behavior expected in the simulation, with denser tetrahedral meshing applied to more anatomically complex areas and quadrilateral elements used in regions where less detail is required.

The system may rely on advanced meshing algorithms that analyze the input medical imaging data to determine the optimal distribution of tetrahedral and quadrilateral elements across the heart. These algorithms may evaluate parameters like surface curvature, mechanical loading, and anatomical complexity to assign the appropriate element type for each region. Additionally, user-defined criteria could guide the meshing process, allowing for adjustments based on specific goals, such as prioritizing accuracy in regions of interest or optimizing computational efficiency for large-scale simulations of heart function. This hybrid meshing approach allows the present methods and systems to balance the need for detailed modeling in critical areas with the computational resources available for efficient simulation.

The process of determining whether regions of the heart should be meshed with tetrahedral or quadrilateral elements may involve several mathematical techniques, including curvature analysis, geometric decomposition, and optimization algorithms. For example, curvature calculations such as Gaussian curvature (K=k₁×k₂) where the product of the two principal curvatures (k₁ and k₂) provide a measure of a given surface bends. Areas with high Gaussian curvature are more likely to require tetrahedral meshing, while regions with low curvature can often be effectively represented with quadrilateral elements. Similarly, mean curvature

$$H = ( \frac{K 1 + K 2}{2} )$$

may be used to identify smoother surfaces, which may be associated with tetrahedral elements.

Feature recognition algorithms may apply principal component analysis (PCA) to decompose the surface into regions based on complexity. By analyzing the eigenvalues of the surface, these algorithms identify regions with large variations, indicating areas that may be suited for tetrahedral elements.

The present systems and methods may evaluate mesh quality by analyzing metrics like aspect ratio and/or determining element distortion (e.g., by computing a Jacobian determinant

$$J = ( \frac{\partial ( x , y , z )}{\partial ( u , v , w )} ) .$$

Poor Jacobian values may be used to indicate an area that is a good candidate for a tetrahedral refinement process.

One or more optimization algorithms (e.g., energy minimization) may be used to optimally distribute the one or more tetrahedral and/or quadrilateral elements. One or more adaptive meshing techniques may be derived from one or more finite element methods to predict stress and strain distribution in different regions. Thus, the present methods and systems may apply tetrahedral elements in regions with higher stress or strain and quadrilateral elements in regions with more uniform mechanical behavior.

In regions of complex geometry, element subdivision can be performed, especially in areas of high curvature, using h-refinement, where the element size (h) is reduced. For example, a large quadrilateral element may be subdivided into smaller tetrahedral elements if curvature exceeds a certain threshold. Shape preservation methods, such as conformal mapping, ensure that the geometry of the heart remains accurate during meshing by preserving angles between elements. Topology optimization may be used to determine the distribution of elements by solving optimization problems to minimize an objective function, (e.g., related to stress or deformation).

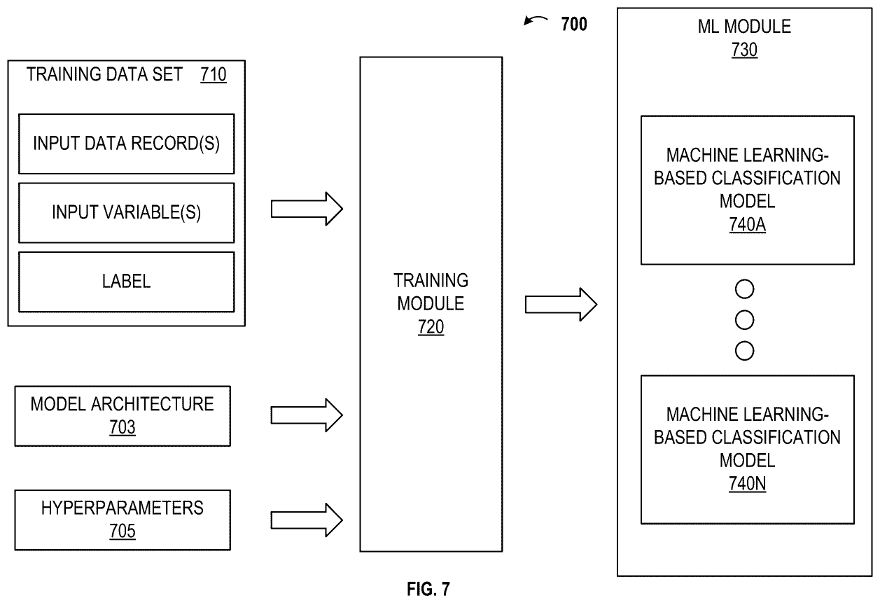

Turning now to FIG. 7, an example method 700 is shown. While FIG. 7 illustrates a training method, it is to be understood that the systems and methods described herein may be implemented via pre-trained model. The method 700 may be performed based on an analysis of one or more training data sets 710 by a training module 720, at least one ML module 730 that is configured to provide one or more of a prediction or a score associated with data records and one or more corresponding variables. The training module 720 may be configured to train and configure the ML module 730 using one or more hyperparameters 705 and a model architecture 703. The one or more hyperparameters 705 may include mesh density, projection tolerance, element aspect ratio, optimization parameters, convergence tolerance, combinations thereof, and the like. The model architecture 703 may comprise a predictive model as described herein. The hyperparameters 705 may comprise a number of neural network layers/blocks, a number of neural network filters (e.g., convolutional filters) in a neural network layer, a number of epochs etc. For text features, a transformer-based encoder model may be used. For audio features, one or more CNN based models may be used. Each set of the hyperparameters 705 may be used to build the model architecture 703, and an element of each set of the hyperparameters 705 may comprise a number of inputs (e.g., data record attributes/variables) to include in the model architecture 703. For example, the first set of hyperparameters may be associated with a first model. The first model may be associated with a first task (e.g., a source task). The first task may comprise population level analysis. The second set of hyperparameters may be associated with a second model. The second model may be associated with a second task (e.g., the target task). In other words, an element of each set of the hyperparameters 705 may indicate that as few as one or as many as all corresponding attributes of the data records and variables are to be used to build the model architecture 703 that is used to train the ML module 730.

The training data set 710 may comprise one or more input data records associated with one or more labels (e.g., a binary label (yes/no, hypo/non-hypo), a multi-class label (e.g., hypo/non/hyper) and/or a percentage value). The label for a given record and/or a given variable may be indicative of a likelihood that the label applies to the given record. A subset of the data records may be randomly assigned to the training data set 710 or to a testing data set. In some implementations, the assignment of data to a training data set or a testing data set may not be completely random. In this case, one or more criteria may be used during the assignment. In general, any suitable method may be used to assign the data to the training or testing data sets, while ensuring that the distributions of yes and no labels are somewhat similar in the training data set and the testing data set.

The training module 720 may train the ML module 730 by extracting a feature set from a plurality of data records (e.g., labeled as yes, hypo/hyper, no for normo) in the training data set 710 according to one or more feature selection techniques. The training module 720 may train the ML module 730 by extracting a feature set from the training data set 710 that includes statistically significant features of positive examples (e.g., labeled as being yes) and statistically significant features of negative examples (e.g., labeled as being no).

The training module 720 may extract a feature set from the training data set 710 in a variety of ways. The training module 720 may perform feature extraction multiple times, each time using a different feature-extraction technique. In an example, the feature sets generated using the different techniques may each be used to generate different machine learning-based classification models 740A-440N. For example, the feature set with the highest quality metrics may be selected for use in training. The training module 720 may use the feature set(s) to build one or more machine learning-based classification models 740A-440N that are configured to indicate whether a particular label applies to a new/unseen data record based on its corresponding one or more variables.

The training data set 710 may be analyzed to determine any dependencies, associations, and/or correlations between features and the yes/no labels in the training data set 710. The identified correlations may have the form of a list of features that are associated with different yes/no labels. The term “feature,” as used herein, may refer to any characteristic of an item of data that may be used to determine whether the item of data falls within one or more specific categories. A feature selection technique may comprise one or more feature selection rules. The one or more feature selection rules may comprise a feature occurrence rule. The feature occurrence rule may comprise determining which features in the training data set 710 occur over a threshold number of times and identifying those features that satisfy the threshold as candidate features.

Two commonly-used retraining approaches are based on initialization and feature extraction. In the initialization approach the whole network is further trained, while in the feature extraction approach the last few fully-connected layers are trained from a random initialization, and other layers remain unchanged. In addition to these two approaches, a third approach may be implemented by combining these two approaches (e.g., the last few fully-connected layers are further trained, and other layers remain unchanged).

A single feature selection rule may be applied to select features or multiple feature selection rules may be applied to select features. The feature selection rules may be applied in a cascading fashion, with the feature selection rules being applied in a specific order and applied to the results of the previous rule. For example, the feature occurrence rule may be applied to the training data set 710 to generate a first list of features. A final list of candidate features may be determined, generated, and/or analyzed according to additional feature selection techniques to determine one or more candidate feature groups (e.g., groups of features that may be used to predict whether a label applies or does not apply). Any suitable computational technique may be used to identify the candidate feature groups using any feature selection technique such as filter, wrapper, and/or embedded methods. One or more candidate feature groups may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable (e.g., yes/no).

As another example, one or more candidate feature groups may be selected according to a wrapper method. A wrapper method may be configured to use a subset of features and train a machine learning model using the subset of features. Based on the inferences that drawn from a previous model, features may be added and/or deleted from the subset. Wrapper methods include, for example, forward feature selection, backward feature elimination, recursive feature elimination, combinations thereof, and the like. As an example, forward feature selection may be used to identify one or more candidate feature groups. Forward feature selection is an iterative method that begins with no feature in the machine learning model. In each iteration, the feature which best improves the model is added until an addition of a new variable does not improve the performance of the machine learning model. As an example, backward elimination may be used to identify one or more candidate feature groups. Backward elimination is an iterative method that begins with all features in the machine learning model. In each iteration, the least significant feature is removed until no improvement is observed on removal of features. Recursive feature elimination may be used to identify one or more candidate feature groups. Recursive feature elimination is a greedy optimization algorithm which aims to find the best performing feature subset. Recursive feature elimination repeatedly creates models and keeps aside (e.g., includes and/or excludes) the best or the worst performing feature at each iteration. Recursive feature elimination constructs the next model with the features remaining until all the features are exhausted. Recursive feature elimination then ranks the features based on the order of their elimination.

As a further example, one or more candidate feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After the training module 720 has generated a feature set(s), the training module 720 may generate one or more machine learning-based classification models 740A-740N based on the feature set(s). A machine learning-based classification model may refer to a complex mathematical model for data classification that is generated using machine-learning techniques. In one example, the machine learning-based classification model 740 may include a map of support vectors that represent boundary features. By way of example, boundary features may be selected from, and/or represent the highest-ranked features in, a feature set. The boundary features may be configured to separate or classify data points into different categories or classes.

The training module 720 may use the feature sets extracted from the training data set 710 to build the one or more machine learning-based classification models 740A-440N for each classification category (e.g., yes, no, hypo/non, hypo/non/hyper). In some examples, the machine learning-based classification models 740A-440N may be combined into a single machine learning-based classification model 740. Similarly, the ML module 730 may represent a single classifier containing a single or a plurality of machine learning-based classification models 740 and/or multiple classifiers containing a single or a plurality of machine learning-based classification models 740.

The extracted features (e.g., one or more candidate features) may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting ML module 730 may comprise a decision rule or a mapping for each candidate feature.

The candidate feature(s) and the ML module 730 may be used to predict whether a label applies to a data record in the testing data set. In one example, the result for each data record in the testing data set includes a confidence level that corresponds to a likelihood or a probability that the one or more corresponding variables are indicative of the label applying to the data record in the testing data set. The confidence level may be a value between zero and one, and it may represent a likelihood that the data record in the testing data set belongs to a yes/no status with regard to the one or more corresponding variables. In one example, when there are two statuses (e.g., yes and no), the confidence level may correspond to a value p, which refers to a likelihood that a particular data record in the testing data set belongs to the first status (e.g., yes). In this case, the value 1−p may refer to a likelihood that the particular data record in the testing data set belongs to the second status (e.g., no). In general, multiple confidence levels may be provided for each data record in the testing data set and for each candidate feature when there are more than two labels. A top performing candidate feature may be determined by comparing the result obtained for each test data record with the known yes/no label for each data record. In general, the top performing candidate feature will have results that closely match the known yes/no labels. The top performing candidate feature(s) may be used to predict the yes/no label of a data record with regard to one or more corresponding variables. For example, a new data record may be determined/received. The new data record may be provided to the ML module 730 which may, based on the top performing candidate feature, classify the label as either applying to the new data record or as not applying to the new data record.

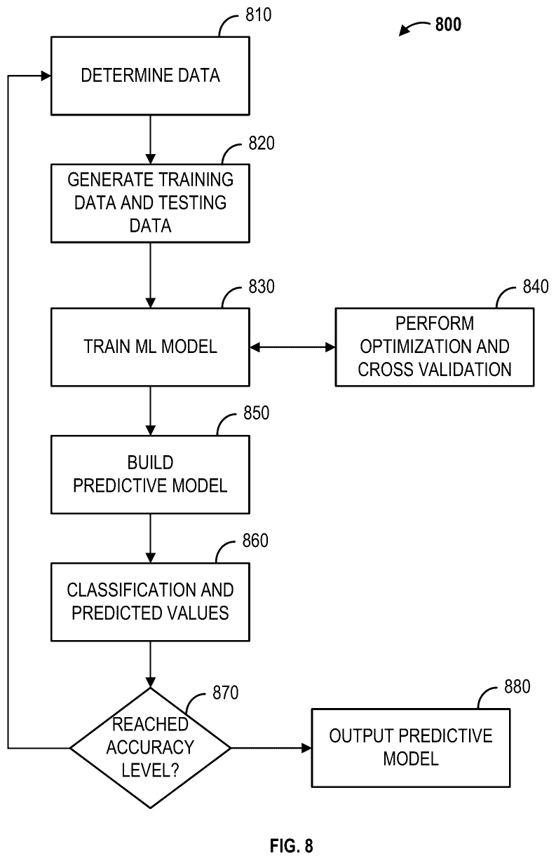

FIG. 8 shows a flowchart illustrating an example training method 800 for generating the ML module 730 using the training module 720 is shown. The training module 720 can implement supervised, unsupervised, and/or semi-supervised (e.g., reinforcement based) machine learning-based classification models 740A-740N. The training module 720 may comprise a data processing module and/or a predictive module. The method 800 illustrated in FIG. 8 is an example of a supervised learning method; variations of this example of training method are discussed below, however, other training methods can be analogously implemented to train unsupervised and/or semi-supervised machine learning models.

The training method 800 may determine (e.g., access, receive, retrieve, etc.) first data records that have been processed by the data processing module at step 810. The first data records may comprise a labeled set of data records. The labels may correspond to a label (e.g., yes or no). The training method 800 may generate, at step 820, a training data set and a testing data set. The training data set and the testing data set may be generated by randomly assigning labeled data records to either the training data set or the testing data set. In some implementations, the assignment of labeled data records as training or testing samples may not be completely random. As an example, a majority of the labeled data records may be used to generate the training data set. For example, 85% of the labeled data records may be used to generate the training data set and 85% may be used to generate the testing data set. The training data set may comprise population data that excludes data associated with a target patient.

The training method 800 may train one or more machine learning models at step 830. In one example, the machine learning models may be trained using supervised learning. In another example, other machine learning techniques may be employed, including unsupervised learning and semi-supervised. The machine learning models trained at 830 may be selected based on different criteria depending on the problem to be solved and/or data available in the training data set. For example, machine learning classifiers can suffer from different degrees of bias. Accordingly, more than one machine learning model can be trained at 830, optimized, improved, and cross-validated at step 840.

For example, a loss function may be used when training the machine learning models at step 830. The loss function may take true labels and predicted outputs as its inputs, and the loss function may produce a single number output. The present methods and systems may implement a mean absolute error, relative mean absolute error, mean squared error and relative mean squared error using the original training dataset without data augmentation.

One or more minimization techniques may be applied to some or all learnable parameters of the machine learning model (e.g., one or more learnable neural network parameters) in order to minimize the loss. For example, the one or more minimization techniques may not be applied to one or more learnable parameters, such as encoder modules that have been trained, a neural network block(s), a neural network layer(s), etc. This process may be continuously applied until some stopping condition is met, such as a certain number of repeats of the full training dataset and/or a level of loss for a left-out validation set has ceased to decrease for some number of iterations. In addition to adjusting these learnable parameters, one or more of the hyperparameters 705 that define the model architecture 703 of the machine learning models may be selected. The one or more hyperparameters 705 may comprise a number of neural network layers, a number of neural network filters in a neural network layer, etc. For example, as discussed above, each set of the hyperparameters 705 may be used to build the model architecture 703, and an element of each set of the hyperparameters 705 may comprise a number of inputs (e.g., data record attributes/variables) to include in the model architecture 703. The element of each set of the hyperparameters 705 comprising the number of inputs may be considered the “plurality of features” as described herein. That is, the cross-validation and optimization performed at step 840 may be considered as a feature selection step. An element of a second set of the hyperparameters 805 may comprise data record attributes for a particular patient. In order to select the best hyperparameters 705, at step 840 the machine learning models may be optimized by training the same using some portion of the training data (e.g., based on the element of each set of the hyperparameters 705 comprising the number of inputs for the model architecture 703). The optimization may be stopped based on a left-out validation portion of the training data. A remainder of the training data may be used to cross-validate. This process may be repeated a certain number of times, and the machine learning models may be evaluated for a particular level of performance each time and for each set of hyperparameters 705 that are selected (e.g., based on the number of inputs and the particular inputs chosen).

A best set of the hyperparameters 705 may be selected by choosing one or more of the hyperparameters 705 having a best mean evaluation of the “splits” of the training data. This function may be called for each new data split, and each new set of hyperparameters 705. A cross-validation routine may determine a type of data that is within the input (e.g., attribute type(s)), and a chosen amount of data (e.g., a number of attributes) may be split-off to use as a validation dataset. A type of data splitting may be chosen to partition the data a chosen number of times. For each data partition, a set of the hyperparameters 705 may be used, and a new machine learning model comprising a new model architecture 703 based on the set of the hyperparameters 705 may be initialized and trained. After each training iteration, the machine learning model may be evaluated on the test portion of the data for that particular split. The evaluation may return a single number, which may depend on the machine learning model's output and the true output label. The evaluation for each split and hyperparameter set may be stored in a table, which may be used to select the optimal set of the hyperparameters 705. The optimal set of the hyperparameters 705 may comprise one or more of the hyperparameters 705 having a highest average evaluation score across all splits.

The training method 800 may select one or more machine learning models to build a predictive model at 850. The predictive model may be evaluated using the testing data set. The predictive model may analyze the testing data set and generate one or more of a prediction or a score at step 860. The one or more predictions and/or scores may be evaluated at step 870 to determine whether they have achieved a desired accuracy level. Performance of the predictive model may be evaluated in a number of ways based on a number of true positives, false positives, true negatives, and/or false negatives classifications of the plurality of data points indicated by the predictive model.

For example, the false positives of the predictive model may refer to a number of times the predictive model incorrectly classified a label as applying to a given data record when in reality the label did not apply. Conversely, the false negatives of the predictive model may refer to a number of times the machine learning model indicated a label as not applying when, in fact, the label did apply. True negatives and true positives may refer to a number of times the predictive model correctly classified one or more labels as applying or not applying. Related to these measurements are the concepts of recall and precision. Generally, recall refers to a ratio of true positives to a sum of true positives and false negatives, which quantifies a sensitivity of the predictive model. Similarly, precision refers to a ratio of true positives a sum of true and false positives. When such a desired accuracy level is reached, the training phase ends and the predictive model (e.g., the ML module 730) may be output at step 880; when the desired accuracy level is not reached, however, then a subsequent iteration of the training method 800 may be performed starting at step 810 with variations such as, for example, considering a larger collection of data records.

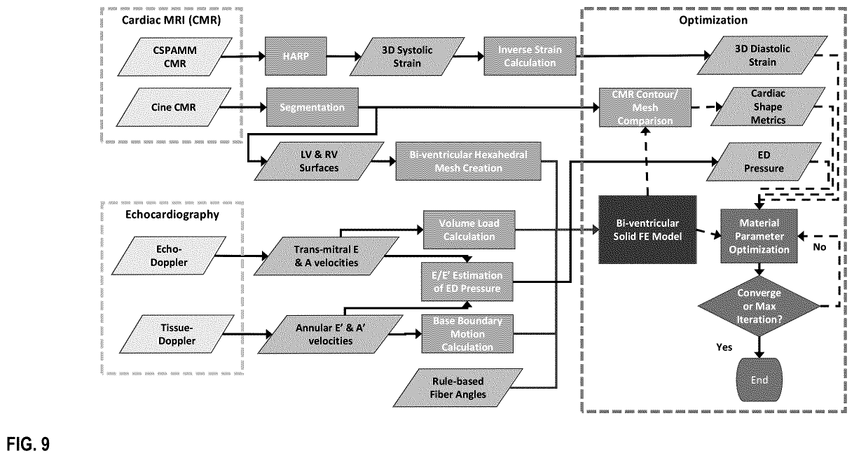

FIG. 9 shows an example system and method. The method begins with input data from both Cardiac MRI (CMR) and Echocardiography to create a detailed and accurate bi-ventricular finite element (FE) model of the heart. CMR techniques like CSPAMM and Cine MRI capture dynamic images of the heart during different phases of the cardiac cycle. These images may be processed through HARP (Harmonic Phase Analysis), which calculates 3D systolic strain, a key measure of how the heart muscle deforms during contraction.

Afterward, the CMR images undergo segmentation, where the left ventricular (LV) and right ventricular (RV) surfaces are isolated, providing the geometric foundation for creating a bi-ventricular hexahedral mesh. This mesh is essential for constructing the FE model that will simulate the heart's mechanical function.

At the same time, Echocardiography data is collected, including Echo-Doppler and Tissue Doppler measurements, which assess blood flow and tissue movement during the cardiac cycle. Specifically, trans-mitral E and A velocities capture how blood flows into the left ventricle, and annular E′ and A′ velocities measure the motion of the mitral valve annulus, both of which provide valuable information for estimating end-diastolic (ED) pressure and calculating volume load on the heart. The system also incorporates base boundary motion, derived from Doppler data, to ensure that the FE model accurately reflects how the base of the heart moves during each cardiac cycle. Additionally, rule-based fiber angles are included, defining the muscle fiber orientation within the heart, which is useful because the direction of the fibers significantly influences heart contraction and relaxation mechanics.

All of this data is integrated to create a bi-ventricular solid FE model. The model simulates the heart's behavior under different loads and pressures, allowing the system to assess its mechanical performance. To ensure the model's accuracy, an optimization process compares the simulation results, including 3D diastolic strain and cardiac shape metrics, against real-world measurements from CMR data. The optimization process also takes into account ED pressure and heart shape metrics, refining the model through material parameter optimization if discrepancies are identified. This iterative optimization continues until the model either converges—accurately matching real heart behavior—or reaches the maximum number of iterations.

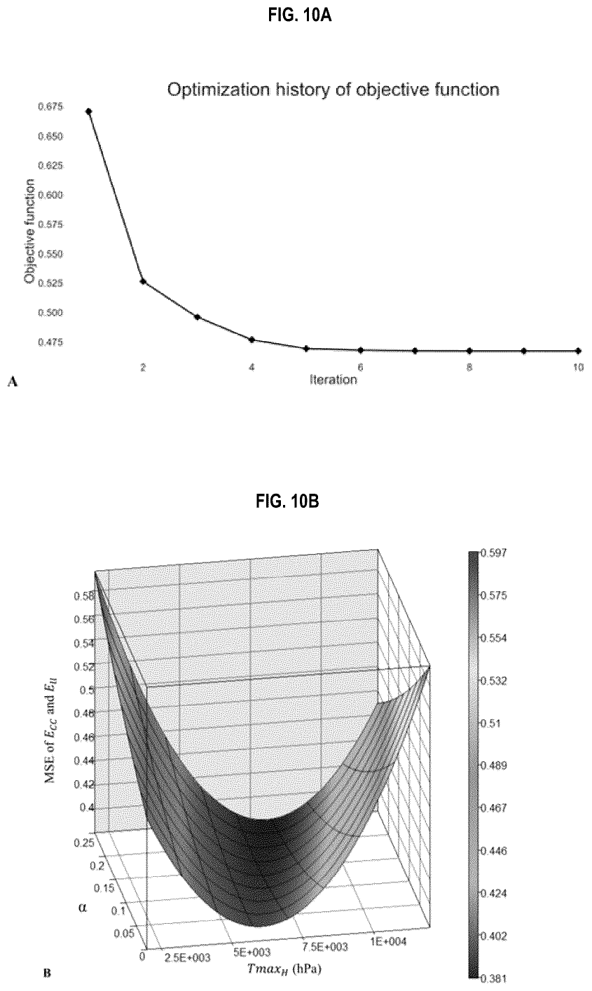

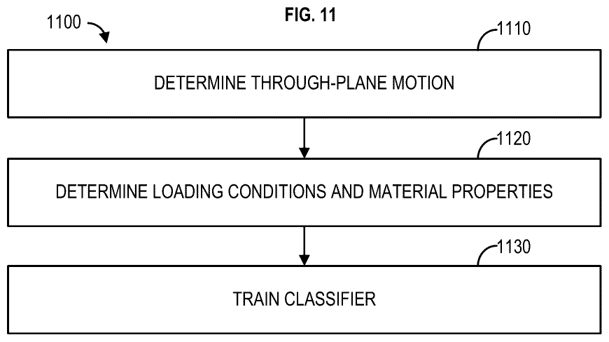

FIG. 10A shows the optimization history of the objective function as part of the iterative process for determining the non-linear stiffness parameters in the finite element (FE) model of the heart, as discussed above. The graph shows the objective function on the vertical axis and the number of iterations on the horizontal axis, tracking how the objective function decreases over successive iterations.

The objective function may be configured to quantify the difference between the simulated strain patterns in the heart (using the FE model) and the actual strain data obtained from Cardiac MRI (CMR) and Echocardiography. Initially, the objective function starts at a high value, around 0.675, indicating a significant discrepancy between the simulated model and the observed strain data. As the optimization progresses, the system adjusts material parameters, such as the non-linear stiffness for the left and right sides of the heart, to reduce this difference.

For example, by the fourth iteration, the objective function has significantly decreased, suggesting that the model has become much closer to matching the actual strain data. After about the sixth iteration, the objective function stabilizes around 0.475, indicating that further changes to the parameters yield minimal improvement. At this point, the system has likely converged, meaning that the optimized parameters for non-linear stiffness of the heart tissue accurately represent the material behavior of one or more ventricles.

FIG. 10B shows an example response surface. The response surface shows the relationship between one or more parameters of the heart tissue model and their impact on the mean squared error (MSE) between the simulated strain and the actual strain data from Cardiac MRI (CMR) and Echocardiography. The response surface illustrates how changes in these parameters affect the model's accuracy, guiding the optimization process for determining non-linear stiffness of the heart. In FIG. 7B, the vertical axis represents the MSE of Ecc and En, which may be, for example, circumferential and longitudinal strain components measured in the heart. For example, a lower MSE indicates a better fit between the model's predicted strain and the actual strain observed from imaging data.

The horizontal axis shows one or more material parameters in the model. For example, T_{maxH} (hPa) may represent a maximum stress or tension experienced in the eart tissue.

For example, α may represent a parameter controlling the non-linear behavior of the heart tissue (e.g., similar to the exponent in the constitutive equation like the Ogden model).

The Ogden Model is widely used to describe the complex, non-linear elasticity of soft biological tissues. The Ogden model is particularly well-suited for materials that exhibit large deformations and non-linear behavior, such as heart tissue. The general form of the Ogden model for isotropic hyperelastic materials is:

$$W - {\sum }_{p = 1}^{N} \frac{{μ}_{p}}{{a}_{p}} ( {λ}_{1}^{{a}_{p}} + {λ}_{2}^{{a}_{p}} + {λ}_{3}^{{a}_{p}} - 3 )$$

where W is the strain energy density function, which represents the stored energy per unit volume due to deformation, μₚ are material parameters that control the stiffness of the material (analogous to shear moduli), αₚ are dimensionless material parameters that control the non-linear response of the material, and λ₁, λ₂, and λ₃, are the principal stretches, representing the ratios of deformed to undeformed lengths in the principal directions.

The strain energy density function (W) represents how much energy is stored in the material due to deformation. It is used to describe the internal energy of the tissue under mechanical loading. Material parameters μₚ and αₚ are specific to the tissue being modeled. They are determined empirically from experiments and define the mechanical properties of the heart tissue, μₚ relates to the stiffness, while αₚ while adjusts the degree of non-linearity in the stress-strain response. The principal stretches λ₁, λ₂, and λ₃ are measures of the amount of stretching or compression in the tissue along three principal axes. They describe how much the material deforms in different directions.

While the above describes use of the Ogden model, it is to be understood the present methods and systems may use any appropriate model.

For a given deformation, the Cauchy stress tensor σ in terms of the principal stretches may be derived from the strain energy function by differentiating W with respect to the stretches. For the Ogden model (although other models may be used), the Cauchy stress tensor σᵢ in the i-th principal direction is:

$${σ}_{i} - {\sum }_{p = 1}^{N} {μ}_{p} {λ}_{i}^{{α}_{p} - 1}$$

where σᵢ is the Cauchy stress in the i-th direction, λᵢ is the principal stretch in the i-th direction, and μₚ and αₚ are the same material parameters from the strain energy function.

The present methods and systems may incorporate the Ogden model (or one or more other appropriate models) and one or more shape metrics. For example, the one or more shape metrics may describe such as the candidate object's overall geometry and/or one or more specific geometries (e.g., the curvature of the ventricular walls, or chamber dilation). These metrics can be incorporated into heart models by combining the Ogden model with finite element analysis (FEA), which uses 3D representations of the heart to account for its shape.

The one or more shape metrics may be integrated into the model through the geometry of the mesh, derived from medical imaging (MRI, CT, or ultrasound), which captures the heart's physical shape and structural details. Boundary conditions may be applied to simulate how the heart moves and pumps blood, affecting stress and strain distribution. The Ogden model or other similarly appropriate models may be adapted to represent different material properties across regions of the heart, like distinguishing between healthy and scarred tissue, and applying these properties according to the heart's shape for better accuracy. Patient-specific models may be generated which determine the heart's shape from imaging data and incorporate that data with the Ogden model to simulate mechanical function.

For example, in a bi-ventricular heart model, the heart's geometry may be captured as a 3D mesh (e.g., the hybrid mesh described herein) that represents the shape, wall thickness, and curvature of the ventricles. The Ogden model can be applied to describe the tissue's response to stress across this geometry, allowing for local variations in material properties based on the tissue's health.

The color gradient in FIG. 7B may indicate error. For example, the color may range from a first shade or color (e.g., red) to a second shade or color (e.g., blue) thereby indicating higher or lower error. For example, lighter shade may indicate higher error values and darker shades indicating lower error values. The vertical axis, representing the mean squared error (MSE) between the simulated and observed strain data, shows how the error varies based on the two parameters on the horizontal axes.

For example, at the top of the surface (corresponding to the lighter shades), the MSE is highest, indicating a poor fit between the model's predictions and the actual strain data. As the surface descends towards the base of the curve (where the darkest shades are located), the MSE decreases, representing a closer match between the simulated and observed data. The darkest region at the bottom of the surface identifies the optimal parameter combination, where the error is minimized, showing that the model has accurately captured the heart's mechanical behavior with respect to the non-linear stiffness parameters.

The present methods and systems may incorporate a trained classifier to analyze this error distribution across the surface. The classifier may be configured to pinpoint the region where the MSE is minimized, identifying the parameter combination that leads to the most accurate prediction of the heart's mechanical behavior. The classifier may be configured to evaluate various combinations of the two parameters, guiding the system toward the darkest region of the surface (lowest error), where the non-linear stiffness parameters for both the left and right ventricles are optimally defined. By doing so, the classifier aids in efficiently locating the optimal area with the lowest error, ensuring that the model closely matches the observed strain data.

FIG. 11 shows an example method 1100. At 1110, through-plane motion associated with a plurality of objects may be determined. The through-plane motion of the plurality of objects may be determined based on image data. The image data may comprise, for example, MRI data, CT data, ultrasound data, PET data, x-ray data, or any other data gathered by any other imaging technology. Through-plane motion refers to the movement of anatomical structures perpendicular to the imaging plane in medical imaging procedures. This type of motion can occur during various imaging modalities such as magnetic resonance imaging (MRI), computed tomography (CT), and ultrasound. Through-plane motion can significantly impact image quality, leading to motion artifacts and potentially compromising diagnostic accuracy. In medical imaging, through-plane motion is determined by assessing the displacement of anatomical structures along the imaging plane's perpendicular direction. Gating techniques may be implemented. Gating techniques involve synchronizing image acquisition with specific physiological events, such as the cardiac cycle or respiratory cycle. By gating image acquisition to specific phases of motion, through-plane motion artifacts can be minimized, enhancing image quality and diagnostic accuracy. Respiratory motion may be corrected. Advanced imaging techniques, such as respiratory motion correction algorithms, are employed to compensate for through-plane motion caused by breathing. These algorithms track respiratory motion during image acquisition and adjust imaging parameters to minimize motion artifacts and improve image clarity. Image reconstruction techniques may be employed. Sophisticated image reconstruction algorithms are utilized to mitigate through-plane motion artifacts during post-processing. These algorithms employ motion correction techniques, such as registration-based methods, to align image data acquired at different time points and reduce motion-related distortions.

At 1120, one or more loading conditions and one or more material properties may be determined. The one or more loading conditions and/or one or more material properties may be associated with one or more mesh representations of one or more physical objects (e.g., one or more organs, muscles, tissues, systems, other body parts, combinations thereof, and the like). As used herein, the term “material property” refers to any description of the physical characteristics of the anatomy described by the geometric mesh in response to physical loads. In addition, as used herein, “material property” also refers to the response to pharmacologic, electrical, magnetic, or heating or cooling interventions. In addition, as used herein, “material property” also refers to any characteristic of anatomy that can be represented as physical changes, whether directly or indirectly through biological changes.

As used herein, the term “loading condition” refers to any description of the physical loads applied to or experienced by the anatomy. In addition, as used herein, the “loading condition” includes any description of pharmacologic, electrical, magnetic, or heating or cooling interventions.

At 1130, a classifier may be trained. The classifier may be configured to determine the physical (e.g., anatomically) feasibility/plausibility of one or more loading conditions, material properties, physical actions, combinations thereof, and the like. For example, the classifier may be configured to determine, based on the one or more loading conditions and/or material properties, whether a modeled bend in a septum of the heart is feasible.

FIG. 12 shows an example method 1200. At 1210, through-plane motion associated with a plurality of objects may be determined. The through-plane motion of the plurality of objects may be determined based on image data. The image data may comprise, for example, MRI data, CT data, ultrasound data, PET data, x-ray data, or any other data gathered by any other imaging technology. Through-plane motion refers to the movement of anatomical structures perpendicular to the imaging plane in medical imaging procedures. This type of motion can occur during various imaging modalities such as magnetic resonance imaging (MRI), computed tomography (CT), and ultrasound. Through-plane motion can significantly impact image quality, leading to motion artifacts and potentially compromising diagnostic accuracy. In medical imaging, through-plane motion is determined by assessing the displacement of anatomical structures along the imaging plane's perpendicular direction. Gating techniques may be implemented. Gating techniques involve synchronizing image acquisition with specific physiological events, such as the cardiac cycle or respiratory cycle. By gating image acquisition to specific phases of motion, through-plane motion artifacts can be minimized, enhancing image quality and diagnostic accuracy. Respiratory motion may be corrected. Advanced imaging techniques, such as respiratory motion correction algorithms, are employed to compensate for through-plane motion caused by breathing. These algorithms track respiratory motion during image acquisition and adjust imaging parameters to minimize motion artifacts and improve image clarity. Image reconstruction techniques may be employed. Sophisticated image reconstruction algorithms are utilized to mitigate through-plane motion artifacts during post-processing. These algorithms employ motion correction techniques, such as registration-based methods, to align image data acquired at different time points and reduce motion-related distortions.

At 1220, one or more loading conditions and one or more material properties may be determined. The one or more loading conditions and/or one or more material properties may be associated with one or more mesh representations of one or more physical objects (e.g., one or more organs, muscles, tissues, systems, other body parts, combinations thereof, and the like). For example, the one or more mesh representations of the plurality of objects may comprise one or more tetrahedral regions and one or more hexahedral regions. As used herein, the term “material property” refers to any description of the physical characteristics of the anatomy described by the geometric mesh in response to physical loads. In addition, as used herein, “material property” also refers to the response to pharmacologic, electrical, magnetic, or heating or cooling interventions. In addition, as used herein, “material property” also refers to any characteristic of anatomy that can be represented as physical changes, whether directly or indirectly through biological changes.

As used herein, the term “loading condition” refers to any description of the physical loads applied to or experienced by the anatomy. In addition, as used herein, the “loading condition” includes any description of pharmacologic, electrical, magnetic, or heating or cooling interventions.

At 1230, a classifier may be trained. The classifier may be configured to determine the physical (e.g., anatomically) feasibility/plausibility of one or more loading conditions, material properties, physical actions, combinations thereof, and the like. For example, the classifier may be configured to determine, based on the one or more loading conditions and/or material properties, whether a modeled bend in a septum of the heart is feasible. The classifier may be trained based on the one or more tetrahedral regions of the one or more mesh representations of plurality of objects and one or more hexahedral regions of the one or more mesh representations of the plurality of objects.

In computational modeling of the heart, both tetrahedral and hexahedral elements may be utilized to discretize the heart's volume for simulation purposes. Tetrahedral elements, characterized by their simplicity with four vertices and four triangular faces, excel in representing irregular shapes and complex geometries. They are particularly valuable in simulations requiring accurate depiction of irregular boundaries, such as in computational fluid dynamics (CFD) studies of blood flow within the heart chambers. However, tetrahedral meshes may necessitate more elements for comparable accuracy to hexahedral meshes, potentially leading to higher computational costs. On the other hand, hexahedral elements, defined by their structured nature with eight vertices and six quadrilateral faces, offer advantages in scenarios where regularity and alignment with the underlying structure are critical, such as modeling muscle fibers within the heart. Despite potentially lower computational costs due to requiring fewer elements, hexahedral meshes might struggle to accurately represent regions with significant curvature or irregular geometries. Thus, a hybrid model comprising both tetrahedral and hexahedral components may be used. This increases the speed and efficiency of the classifier, striking a balance between accuracy and computational efficiency in capturing the intricate behavior of the heart.

The method may further comprise determining several parameters of the object, including determining one or more cavity volumes, regional strains, wall thickenings, geometries, contours, and pressures associated with it. These measurements collectively provide a comprehensive understanding of the object's characteristics and behavior. The cavity volumes offer insights into the internal spaces within the object, while regional strains provide information about its structural integrity and deformation patterns. Wall thickenings indicate variations in thickness, which could be indicative of pathological conditions or normal physiological variations. Geometries encompass the overall shape and dimensions of the object, aiding in visual representation and analysis. Contours further delineate specific features or boundaries, aiding in detailed characterization. Finally, pressures provide crucial data on internal forces or fluid dynamics, aiding in the assessment of functional aspects and potential physiological implications of the candidate object. This multi-faceted approach allows for a comprehensive evaluation, essential for accurate diagnosis, research, or design considerations depending on the context of the object under study.

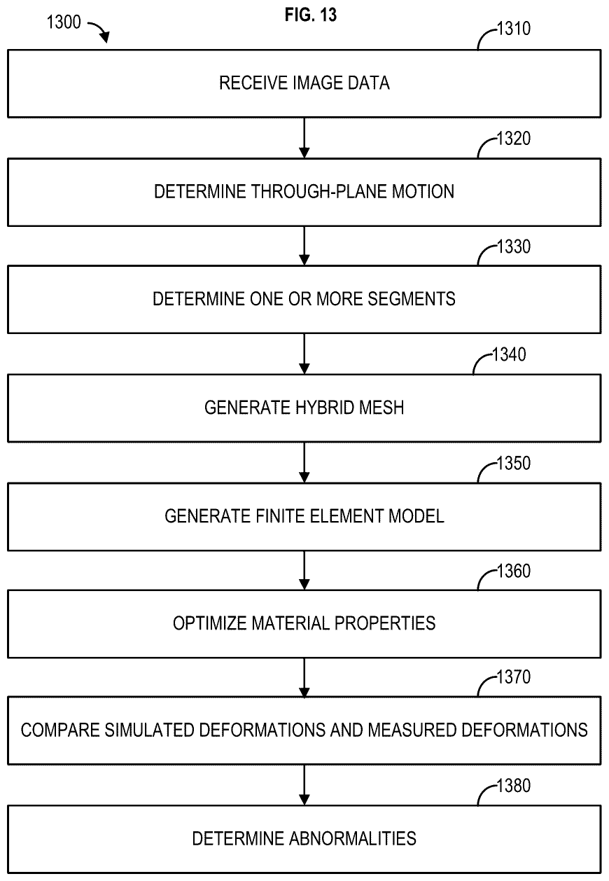

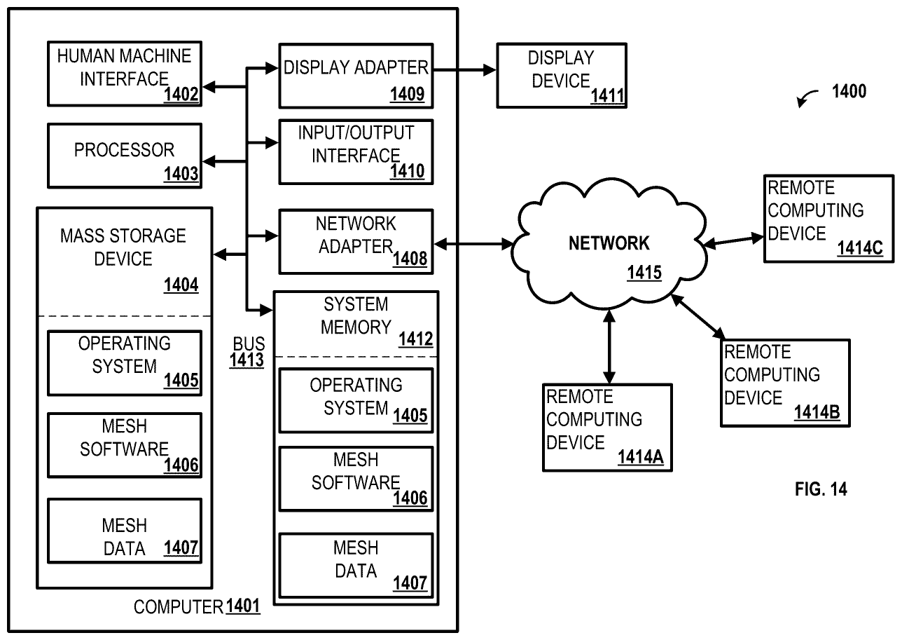

FIG. 13 shows an example method 1300. At 1310, medical image data may be received. For example, the media image data may comprise MRI data, CT data, echocardiography data, combinations thereof, and the like. For example, the medical imaging data may comprise one or more of: receiving cardiac imaging data using Cardiac Magnetic Resonance Imaging (CMR) receiving 3D systolic strain measurements obtained via Harmonic Phase Analysis (HARP), receiving echocardiographic data, including Echo-Doppler and Tissue Doppler measurements, receiving information related to blood flow velocities, tissue motion, end-diastolic pressure, and volume load during the cardiac cycle. At 1320, through-plane motion of the candidate object may be determined.

At 1330, one or more segments of the candidate object may be determined. For example, the one or more segments of the candidate object may be determined based on the medical imaging data associated with the candidate object. For example, the one or more segments of the candidate object may comprise one or more regions of the candidate object, one or more parts of the candidate object, one or more surfaces of the candidate object, one or more structures of the candidate object, combinations thereof, and the like. For example, in the case of the heart, the one or more segments may comprise a left ventricle (LV) segment and a right ventricle (RV) segment. The one or more segments may be associated with one or more materials properties (e.g., stress, strain, stiffness, combinations thereof, and the like).

At 1340, a hybrid mesh representation of the object may be determined. For example, the hybrid mesh representation of the candidate object may be determined based on the one or more segments of the candidate object. The hybrid mesh representation may be generated based on one or more shape metrics associated with the candidate object. Generating the hybrid mesh representation of the candidate object may comprise defining rule-based fiber angles for the muscle fibers in both the left and right ventricles to capture the anisotropic properties of the candidate object. Generating the hybrid mesh representation of the candidate object may comprise segmenting CMR data to isolate the left ventricular (LV) and right ventricular (RV) surfaces and creating a bi-ventricular hexahedral mesh based on the segmented surfaces.

At 1350, a finite element model of the candidate object may be determined. For example, the finite element model of the candidate object may be determined based on the hybrid mesh representation of the candidate object, the one or more material properties, and/or the one or more shape metrics. For example, the finite element model may be generated based on the through-plane motion of the candidate object. Generating the finite element model may comprise creating a bi-ventricular finite element model that simulates the mechanical response of the candidate object under different physiological conditions, using the CMR data, echocardiographic data, and mesh to simulate the candidate object's behavior under volume load and pressure conditions, applying an initial constitutive model to define the material properties of the candidate object.

At 1360, one or more material parameters and one or more shape metrics associated with the finite element model of the candidate object may be optimized. Optimization may include optimizing one or more material parameters, including stiffness and non-linear elasticity, for the left and right sides of the heart by adjusting the parameters iteratively within the finite element model until the simulated deformation patterns converge with the actual observed strain data, wherein the parameters are optimized to describe the non-linear stiffness of the left ventricle and right ventricle tissue based on their respective mechanical demands.

At 1370, one or more simulated deformations associated with the finite element model of the candidate object may be compared with one or more measured deformations and/or one more standard deformations (e.g., normal deformations).

At 1380, one or more abnormalities may be determined. Determining the one or more abnormalities may comprise generating a response surface associated with the

The method may comprise performing an inverse strain calculation to compare simulated heart deformations with actual 3D systolic strain data obtained from the CMR. The method may further comprise identifying discrepancies between the simulated and actual deformation patterns. The method may further comprise the left and right sides of the heart by adjusting parameters iteratively within the finite element model until one or more simulated deformation patterns converge with actual observed strain data, wherein the one or more parameters are optimized to describe a non-linear stiffness of left ventricle tissue and right ventricle tissue based on respective mechanical properties. The method may further comprise outputting the optimized material parameters for the non-linear stiffness of the left and right sides of the heart, wherein the left ventricle and right ventricle are characterized by different non-linear stiffness parameters that reflect the distinct mechanical properties of each side of the heart.

FIG. 14 shows a system 1400 for object modeling and classification. Any device and/or component described herein may be a computer 1401 as shown in FIG. 14. The computer 1401 may comprise one or more processors 1403, a system memory 1412, and a bus 1413 that couples various components of the computer 1401 including the one or more processors 1403 to the system memory 1412. In the case of multiple processors 1403, the computer 1401 may utilize parallel computing. The bus 1413 may comprise one or more of several possible types of bus structures, such as a memory bus, memory controller, a peripheral bus, an accelerated graphics port, and a processor or local bus using any of a variety of bus architectures.

The computer 1401 may operate on and/or comprise a variety of computer-readable media (e.g., non-transitory). Computer-readable media may be any available media that is accessible by the computer 1401 and comprises, non-transitory, volatile, and/or non-volatile media, removable and non-removable media. The system memory 1412 has computer-readable media in the form of volatile memory, such as random access memory (RAM), and/or non-volatile memory, such as read-only memory (ROM). The system memory 1412 may store data such as ERG data 1407 and/or program modules such as operating system 1405 and ERG software 1406 that are accessible to and/or are operated on by the one or more processors 1403.

The computer 1401 may also comprise other removable/non-removable, volatile/non-volatile computer storage media. The mass storage device 1404 may provide non-volatile storage of computer code, computer-readable instructions, data structures, program modules, and other data for the computer 1401. The mass storage device 1404 may be a hard disk, a removable magnetic disk, a removable optical disk, magnetic cassettes or other magnetic storage devices, flash memory cards, CD-ROM, digital versatile disks (DVD) or other optical storage, random access memories (RAM), read-only memories (ROM), electrically erasable programmable read-only memory (EEPROM), and the like.

Any number of program modules may be stored on the mass storage device 1404. An operating system 1405 and ERG software 1406 may be stored on the mass storage device 1404. One or more of the operating system 1405 and ERG software 1406 (or some combination thereof) may comprise program modules and the ERG software 1406. ERG data 1407 may also be stored on the mass storage device 1404. ERG data 1407 may be stored in any of one or more databases known in the art. The databases may be centralized or distributed across multiple locations within the network 1415.

A user may enter commands and information into the computer 1401 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 1403 via a human-machine interface 1402 that is coupled to the bus 1413, but may be connected by other interface and bus structures, such as a parallel port, game port, an IEEE 1494 Port (also known as a Firewire port), a serial port, network adapter 1408, and/or a universal serial bus (USB).

A display device 1411 may also be connected to the bus 1413 via an interface, such as a display adapter 1409. It is contemplated that the computer 1401 may have more than one display adapter 1409 and the computer 1401 may have more than one display device 1411. A display device 1411 may be a monitor, an LCD (Liquid Crystal Display), a light-emitting diode (LED) display, a television, a smart lens, smart glass, and/or a projector. In addition to the display device 1411, other output peripheral devices may comprise components such as speakers (not shown) and a printer (not shown) which may be connected to the computer 1401 via Input/Output Interface 1410. Any step and/or result of the methods may be output (or caused to be output) in any form to an output device. Such output may be any form of visual representation, including, but not limited to, textual, graphical, animation, audio, tactile, and the like. The display 1411 and computer 1401 may be part of one device, or separate devices.

The computer 1401 may operate in a networked environment using logical connections to one or more remote computing devices 1414A,B,C. A remote computing device 1414A,B,C may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network nodes, and so on. Logical connections between the computer 1401 and a remote computing device 1414A,B,C may be made via a network 1415, such as a local area network (LAN) and/or a general wide area network (WAN). Such network connections may be through a network adapter 1408. A network adapter 1408 may be implemented in both wired and wireless environments. Such networking environments are conventional and commonplace in dwellings, offices, enterprise-wide computer networks, intranets, and the Internet.

Application programs and other executable program components such as the operating system 1405 are shown herein as discrete blocks, although it is recognized that such programs and components may reside at various times in different storage components of the computing device 1401, and are executed by the one or more processors 1403 of the computer 1401. An implementation of ERG software 1406 may be stored on or sent across some form of computer-readable media. Any of the disclosed methods may be performed by processor-executable instructions embodied on computer-readable media.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

For purposes of illustration, application programs and other executable program components are illustrated herein as discrete blocks, although it is recognized that such programs and components can reside at various times in different storage components. An implementation of the described methods can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

What is claimed is:

1. A method comprising:
determining, by a computing device, based on medical imaging data received from one or more medical imaging devices, through-plane motion associated with a plurality of objects;
determining, based on the through-plane motion associated with the plurality of objects, one or more loading conditions of one or more mesh representations of the plurality of objects and one or more material properties of the one or more mesh representations of the plurality of objects; and
training, based on the one or more loading conditions of the one or more mesh representations of the plurality of objects and the one or more material properties of the one or more mesh representations of the plurality of objects, a classifier configured to classify one or more material properties associated with a candidate object as biologically plausible or implausible.

2. The method of claim 1, wherein the medical imaging data comprises one or more of:
computed tomography data, magnetic resonance imaging data, ultrasound data, x-ray data, or positron-emission tomography data.

3. The method of claim 1, wherein determining the through-plane motion associated with the plurality of objects comprises measuring movement of one or more anatomical structures perpendicular to an imaging plane in one or more medical imaging procedures.

4. The method of claim 1, further comprising:
determining one or more cavity volumes associated with the candidate object;
determining one or more regional strains associated with the candidate object;
determining one or more wall thickenings associated with the candidate object;
determining one or more geometries associated with the candidate object;
determining one or more contours of the candidate object; and
determining one or more pressures associated with the candidate object.

5. The method of claim 1, further comprising performing a model calibration.

6. The method of claim 5, wherein performing the model calibration comprises:
defining an objective relation to model one or more difference metrics; and
minimizing the one or more difference metrics.

7. The method of claim 1, further comprising:
receiving one or more user inputs; and
based on receiving the one or more user inputs, outputting a mesh representation of the candidate object.

8. One or more non-transitory computer-readable media storing processor executable instructions thereon, which, when executed by at least one processor cause the at least one processor to:
determine, based on medical imaging data received from one or more medical imaging devices, through-plane motion associated with a plurality of objects;
determine, based on the through-plane motion associated with the plurality of objects, one or more loading conditions of one or more mesh representations of the plurality of objects and one or more material properties of the one or more mesh representations of the plurality of objects; and
train, based on the one or more loading conditions of the one or more mesh representations of the plurality of objects and the one or more material properties of the one or more mesh representations of the plurality of objects, a classifier configured to classify through-plane motion associated with a candidate object as biologically plausible or implausible.

9. The one or more non-transitory computer-readable media of claim 8, wherein the medical imaging data comprises one or more of: computed tomography data, magnetic resonance imaging data, ultrasound data, x-ray data, or positron-emission tomography data.

10. The one or more non-transitory computer-readable media of claim 8, wherein the processor executable instructions that, when executed by the at least one processor, cause the at least one processor to determine the through-plane motion associated with the plurality of objects further cause the at least one processor to measure movement of one or more anatomical structures perpendicular to an imaging plane in one or more medical imaging procedures.

11. The one or more non-transitory computer-readable media of claim 8, wherein the processor executable instructions, when executed by the at least one processor, further cause the at least one processor to:
determine one or more cavity volumes associated with the candidate object;
determine one or more regional strains associated with the candidate object;
determine one or more wall thickenings associated with the candidate object;
determine one or more geometries associated with the candidate object;
determine one or more contours of the candidate object; and
determine one or more pressures associated with the candidate object.

12. The one or more non-transitory computer-readable media of claim 8, wherein the processor executable instructions, when executed by the at least one processor, further cause the at least one processor to perform a model calibration.

13. The one or more non-transitory computer-readable media of claim 12, wherein the processor executable instructions that, when executed by the at least one processor, cause the at least one processor to perform the model calibration, further cause the at least one processor to:
define an objective relation to model one or more difference metrics; and
minimize the one or more difference metrics.

14. The one or more non-transitory computer-readable media of claim 8, wherein the processor executable instructions, when executed by the at least one processor, further cause the at least one processor to:
receiving one or more user inputs; and
based on receiving the one or more user inputs, outputting a mesh representation of the candidate object.

15. A method comprising:
determining, by a computing device, based on medical imaging data received from one or more medical imaging devices, through-plane motion associated with a plurality of objects;
determining, based on the through-plane motion associated with the plurality of objects, one or more loading conditions of one or more mesh representations of a plurality of objects and one or more material properties of the one or more mesh representations of the plurality of objects, wherein the one or more mesh representations of the plurality of objects comprise one or more tetrahedral regions and one or more hexahedral regions; and
training, based on the one or more tetrahedral regions of the one or more mesh representations of the plurality of objects and one or more hexahedral regions of the one or more mesh representations of the plurality of objects, a classifier configured to classify a biological plausibility of one or more candidate tetrahedral regions and one or more candidate hexahedral regions.

16. The method of claim 15, wherein the medical imaging data comprises one or more of:
computed tomography data, magnetic resonance imaging data, ultrasound data, x-ray data, or positron-emission tomography data.

17. The method of claim 15, wherein determining the through-plane motion associated with the plurality of objects comprises measuring movement of one or more anatomical structures perpendicular to an imaging plane in one or more medical imaging procedures.

18. The method of claim 15, further comprising:
determining one or more cavity volumes associated with a candidate object;
determining one or more regional strains associated with the candidate object;
determining one or more wall thickenings associated with the candidate object;
determining one or more geometries associated with the candidate object;
determining one or more contours of the candidate object; and
determining one or more pressures associated with the candidate object.

19. The method of claim 15, further comprising performing a model calibration.

20. The method of claim 19, wherein performing the model calibration comprises:
defining an objective relation to model one or more difference metrics; and
minimizing the one or more difference metrics.

21. The method of claim 15, further comprising:
receiving one or more user inputs; and
based on receiving the one or more user inputs, outputting a mesh representation of a candidate object.

22. An apparatus comprising:
one or more processors; and
memory storing processor executable instructions that, when executed by the one or more processors, cause the apparatus to:
determine, based on medical imaging data, through-plane motion associated with a plurality of objects;
determine, based on the through-plane motion associated with the plurality of objects, one or more loading conditions of one or more mesh representations of the plurality of objects and one or more material properties of the one or more mesh representations of the plurality of objects, wherein the one or more mesh representations of the plurality of objects comprise one or more tetrahedral regions and one or more hexahedral regions; and
train, based on the one or more tetrahedral regions of the one or more mesh representations of the plurality of objects and one or more hexahedral regions of the one or more mesh representations, a classifier configured to classify a biological plausibility of one or more candidate tetrahedral regions and one or more candidate hexahedral regions.

23. The apparatus of claim 22, wherein the medical imaging data comprises one or more of: computed tomography data, magnetic resonance imaging data, ultrasound data, x-ray data, or positron-emission tomography data.

24. The apparatus of claim 22, wherein the processor executable instructions that, when executed by the one or more processors, cause the one or more processors to determine the through-plane motion associated with the plurality of objects further cause the one or more processors to measure movement of one or more anatomical structures perpendicular to an imaging plane in one or more medical imaging procedures.

25. The apparatus of claim 22, wherein the processor executable instructions, when executed by the one or more processors, further cause the one or more processors to:
determine one or more cavity volumes associated with a candidate object;
determine one or more regional strains associated with the candidate object;
determine one or more wall thickenings associated with the candidate object;
determine one or more geometries associated with the candidate object;
determine one or more contours of the candidate object; and
determine one or more pressures associated with the candidate object.

26. The apparatus of claim 22, wherein the processor executable instructions, when executed by the one or more processors, further cause the one or more processors to perform a model calibration.

27. The apparatus of claim 26, wherein the processor executable instructions that, when executed by the one or more processors, cause the one or more processors to perform the model calibration, further cause the one or more processors to:
define an objective relation to model one or more difference metrics; and
minimize the one or more difference metrics.

28. A method comprising:
receiving medical image data associated with a candidate object;
determining, based on the medical image data associated with the candidate object, through-plane motion of the candidate object;
determining, based on the medical image data associated with the candidate object, one or more segments of the candidate object;
generating, based on the one or more segments of the candidate object, a hybrid mesh representation of the candidate object;
generating, based on the hybrid mesh representation of the candidate object and the through-plane motion of the candidate object, a finite element model of the candidate object;
optimizing, based on the finite element model of the candidate object, one or more material parameters and one or more shape metrics associated with the finite element model of the candidate object;
comparing one or more simulated deformations associated with the finite element model of the candidate object with one or more measured deformations; and
determining, based on the optimization, one or more abnormalities associated with the candidate object.

29. The method of claim 28 wherein receiving the medical image data comprises one or more of: receiving cardiac imaging data using Cardiac Magnetic Resonance Imaging (CMR) receiving 3D systolic strain measurements obtained via Harmonic Phase Analysis (HARP), receiving echocardiographic data, including Echo-Doppler and Tissue Doppler measurements, receiving information related to blood flow velocities, tissue motion, end-diastolic pressure, and volume load during a cardiac cycle.

30. The method of claim 28, wherein generating the hybrid mesh representation comprises defining rule-based fiber angles for muscle fibers in both a left ventricle and right ventricle to capture anisotropic properties of the candidate object.

31. The method of claim 28, wherein generating the hybrid mesh representation comprises segmenting cardiac magnetic resonance (CMR) data to isolate left ventricular (LV) and right ventricular (RV) surfaces and creating a bi-ventricular hexahedral mesh based on one or more segmented surfaces.

32. The method of claim 28, wherein generating the finite element model comprises creating a bi-ventricular finite element model that simulates a mechanical response of the candidate object under different physiological conditions, using cardiac magnetic resonance (CMR) data, echocardiographic data, and mesh to simulate a candidate object's behavior under volume load and pressure conditions, applying an initial constitutive model to define one or more material properties of the candidate object.

33. The method of claim 28, further comprising:
performing an inverse strain calculation to compare simulated deformations with actual 3D systolic strain data obtained from a cardiac magnetic resonance (CMR) device; and
and identifying discrepancies between the simulated and actual deformation patterns.

34. The method of claim 28, further comprising optimizing one or more parameters, including stiffness and non-linear elasticity, for a left ventricle and a right ventricle of the candidate object by adjusting parameters iteratively within a finite element model until one or more simulated deformation patterns converge with actual observed strain data, wherein the one or more parameters are optimized to describe a non-linear stiffness of left ventricle tissue and right ventricle tissue based on respective mechanical properties.

35. The method of claim 34, further comprising outputting the optimized material parameters for a non-linear stiffness of a left ventricle and a right ventricle, wherein the left ventricle and right ventricle are characterized by different non-linear stiffness parameters that reflect one or more mechanical properties of each side of a candidate object.
